SEQUENCE LISTING (39 sequences)

SEQ ID NO: 1 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 220 aa, synthetic construct
  UNSURE 40: note = S or E or G
  UNSURE 41: note = H or D
  UNSURE 69: note = Y or H
  UNSURE 71: note = S or R
  UNSURE 129: note = D or E
  UNSURE 131: note = L or M
PCPAPELLGGPSVFLFPPKPKDTLMISRTPEVTCVVVDVXXEDPEVKFNWYVDGVEVHNAKTKPREEQXNXTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 220 aa, synthetic construct
  UNSURE 129: note = D or E
  UNSURE 131: note = L or M
PCPAPELLGGPSVFLFPPKPKDTLMISRTPEVTCVVVDVEDEDPEVKFNWYVDGVEVHNAKTKPREEQHNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 220 aa, synthetic construct
  UNSURE 129: note = D or E
  UNSURE 131: note = L or M
PCPAPELLGGPSVFLFPPKPKDTLMISRTPEVTCVVVDVGDEDPEVKFNWYVDGVEVHNAKTKPREEQDNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 220 aa, synthetic construct
  UNSURE 129: note = D or E
  UNSURE 131: note = L or M
PCPAPELLGGPSVFLFPPKPKDTLMISRTPEVTCVVVDVDDEDPEVKFNWYVDGVEVHNAKTKPREEQYNRTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 330 aa, synthetic construct
  UNSURE 150: note = S or E or G
  UNSURE 151: note = H or D
  UNSURE 179: note = Y or H
  UNSURE 181: note = S or R
  UNSURE 239: note = D or E
  UNSURE 241: note = L or M
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVXXEDPEVKFNWYVDGVEVHNAKTKPREEQXNXTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 107 aa, Homo sapiens [sequence omitted]

SEQ ID NO: 10 - protein, 105 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 11 - DNA, 62 bp, synthetic construct
  source 1..62: mol_type = other DNA
atactattcctggtagcaaccgctacaggagtacattccgagcccaaatcttgtgacaaaac

SEQ ID NO: 12 - DNA, 78 bp, synthetic construct
  source 1..78: mol_type = other DNA
gctcgtggtaccgcgcgcctaaatgggttgaaccatgggctggtcctgcatcatactattcctggtagcaaccgctac

SEQ ID NO: 13 - DNA, 43 bp, synthetic construct
  source 1..43: mol_type = other DNA
accggaaccgcctcctgaacctccacctttacccggggacagg

SEQ ID NO: 14 - protein, 31 aa, synthetic construct
GGAGGCGGTTCCGGTAATGCTGTGGGCCAGG

SEQ ID NO: 15 - DNA, 30 bp, synthetic construct
  source 1..30: mol_type = other DNA
gctcgtggatccctaacgtggcttcttctg

SEQ ID NO: 16 - DNA, 40 bp, synthetic construct
  source 1..40: mol_type = other DNA
gctcgtggtaccgcgcgcctaaatgggttgaaccatgggc

SEQ ID NO: 17 - DNA, 40 bp, synthetic construct
  source 1..40: mol_type = other DNA
tggtcctgcatcatactattcctggtagcaaccgctacag

SEQ ID NO: 18 - DNA, 40 bp, synthetic construct
  source 1..40: mol_type = other DNA
gagtacattccgagcccaaatcttgtgacaaaactcacac

SEQ ID NO: 19 - DNA, 40 bp, synthetic construct
  source 1..40: mol_type = other DNA
atgcccaccgtgcccagcacctsawswsswkgrsgrwccg

SEQ ID NO: 20 - DNA, 40 bp, synthetic construct
  source 1..40: mol_type = other DNA
tcagtcttcctcttccccccaaaacccaaggacaccctca

SEQ ID NO: 21 - DNA, 40 bp, synthetic construct
  source 1..40: mol_type = other DNA
tgatctcccggacccctgaggtcacatgcgtggtggtgga

SEQ ID NO: 22 - DNA, 40 bp, synthetic construct
  source 1..40: mol_type = other DNA
cgtgrrsbmcgaagaccctgaggtcaagttcaactggtac

SEQ ID NO: 23 - DNA, 40 bp, synthetic construct
  source 1..40: mol_type = other DNA
gtggacggcgtggaggtgcataatgccaagacaaagccgc

SEQ ID NO: 24 - DNA, 40 bp, synthetic construct
  source 1..40: mol_type = other DNA
gggaggagcagyacaacmrcacgtaccgtgtggtcagcgt

SEQ ID NO: 25 - DNA, 40 bp, synthetic construct
  source 1..40: mol_type = other DNA
cctcaccgtcctgcaccaggactggctgaatggcaaggag

SEQ ID NO: 26 - DNA, 40 bp, synthetic construct
  source 1..40: mol_type = other DNA
tacaagtgcaaggtctccaacaaagmsctcccagccccca

SEQ ID NO: 27 - DNA, 40 bp, synthetic construct
  source 1..40: mol_type = other DNA
tcgagaaaaccatctccaaagccaaagggcagcctcgaga

SEQ ID NO: 28 - DNA, 40 bp, synthetic construct
  source 1..40: mol_type = other DNA
aatagtatgatgcaggaccagcccatggttcaacccattt

SEQ ID NO: 29 - DNA, 40 bp, synthetic construct
  source 1..40: mol_type = other DNA
tttgggctcggaatgtactcctgtagcggttgctaccagg

SEQ ID NO: 30 - DNA, 40 bp, synthetic construct
  source 1..40: mol_type = other DNA
gtgctgggcacggtgggcatgtgtgagttttgtcacaaga

SEQ ID NO: 31 - DNA, 40 bp, synthetic construct
  source 1..40: mol_type = other DNA
ggggggaagaggaagactgacggwycsycmwsswswtsag

SEQ ID NO: 32 - DNA, 40 bp, synthetic construct
  source 1..40: mol_type = other DNA
ctcaggggtccgggagatcatgagggtgtccttgggtttt

SEQ ID NO: 33 - DNA, 40 bp, synthetic construct
  source 1..40: mol_type = other DNA
cagggtcttcgkvsyycacgtccaccaccacgcatgtgac

SEQ ID NO: 34 - DNA, 40 bp, synthetic construct
  source 1..40: mol_type = other DNA
tgcacctccacgccgtccacgtaccagttgaacttgacct

SEQ ID NO: 35 - DNA, 40 bp, synthetic construct
  source 1..40: mol_type = other DNA
gykgttgtrctgctcctcccgcggctttgtcttggcatta

SEQ ID NO: 36 - DNA, 40 bp, synthetic construct
  source 1..40: mol_type = other DNA
cctggtgcaggacggtgaggacgctgaccacacggtacgt

SEQ ID NO: 37 - DNA, 40 bp, synthetic construct
  source 1..40: mol_type = other DNA
ttggagaccttgcacttgtactccttgccattcagccagt

SEQ ID NO: 38 - DNA, 40 bp, synthetic construct
  source 1..40: mol_type = other DNA
tttggagatggttttctcgatgggggctgggagskctttg

SEQ ID NO: 39 - DNA, 40 bp, synthetic construct
  source 1..40: mol_type = other DNA
gcagggtgtacacctgtggttctcgaggctgccctttggc

(19) United States
(12) Patent Application Publication
(10) Pub. No.: US 2025/0188186 A1
(43) Pub. Date: Jun. 12, 2025

(54) PH-SELECTIVE ANTIBODY FC DOMAINS

(71) Applicant: BOARD OF REGENTS, THE UNIVERSITY OF TEXAS SYSTEM, Austin, TX (US)

(72) Inventors: Jennifer MAYNARD, Austin, TX (US); Yutong LIU, Austin, TX (US); Annalee NGUYEN, Austin, TX (US)

(73) Assignee: BOARD OF REGENTS, THE UNIVERSITY OF TEXAS SYSTEM, Austin, TX (US)

(21) Appl. No.: 18/718,241

(22) PCT Filed: Dec. 9, 2022
(86) PCT No.: PCT/US2022/081256

§ 371 Date: Jun. 10, 2024

Related U.S. Application Data
(60) Provisional application No. 63/288,241, filed on Dec. 10, 2021.

(51) Int. Cl. C07K 16/32; A61P 35/00

(52) U.S. Cl. CPC C07K 16/32; A61P 35/00; C07K 2317/14; C07K 2317/522; C07K 2317/524; C07K 2317/526; C07K 2317/53; C07K 2317/732; C07K 2317/92

(57) ABSTRACT

Recombinant polypeptides are provided that comprise pH-selective mammalian IgG1 Fc domains that selectively binds to FcγRIIIa in an acidic environment. The pH-selective mammalian IgG1 Fc domain has reduced affinity for FcγRIIIa at neutral pH as compared to a wild-type mammalian IgG1 Fc domain while having equivalent affinity for FcγRIIIa at pH 6.5 as compared to a wild-type mammalian IgG1 Fc domain. Also provided are recombinant polypeptides comprising the pH-selective mammalian IgG1 Fc domains as well as methods of making and using such recombinant polypeptides.

### REFERENCE TO RELATED APPLICATIONS

The present application claims the priority benefit of U.S. provisional application No. 63/288,241, filed Dec. 10, 2021, the entire contents of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Nov. 9, 2022, is named UTFBP1297WO_ST26.xml and is 44,040 bytes in size.

### BACKGROUND

### 1. Field

The present invention relates generally to the fields of medicine and immunology. More particularly, it concerns antibody design and antibody-based reagents and therapeutics.

### 2. Description of Related Art

Antibody therapeutics have revolutionized cancer treatments by specific recognition of a tumor-associated antigen through the Fab binding site, with protection often mediated by Fc recruitment of immune cells. However, since the tumor-associated molecules targeted can also be present on healthy tissues, many antibody therapeutics exhibit undesirable side effects due to immune activation at non-disease sites. These “on target, off tumor” effects have been reported for a number of monoclonal antibody therapies. For example, the anti-VEGF Bevacizumab disrupts tumor angiogenesis during treatment of lung, kidney, breast, brain and colorectal cancers, but also causes proteinuria in ≤63% of patients and hypertension in ≤36% of patients (Eremina et al., 2008). For the anti-EGFR Cetuximab, approved for treatment of colorectal and skin cancers, various skin disorders arise in a high percentage of patients (Holcmann & Sibilia, 2015). During treatment of Her2+ breast cancer with the anti-Her2 Trastuzumab, clinical results have shown a clear correlation between treatment and impairment of the left ventricular ejection fraction (Piccart-Gebhart et al., 2005; Force et al., 2007), resulting in cardiac dysfunction. These complications can lead to a reduced tolerance for and even discontinuation of therapy.

In addition to expressing tumor-associated antigens, tumors also alter their local tissue environments, which presents opportunities for tumor targeting via characteristics orthogonal to antigen specificity. For example, matrix metalloproteases degrade extracellular matrix components to support tumor invasion into surrounding tissues. Accordingly, matrix metalloprotease inhibitors are progressing as anti-metastatic agents in clinical trials (Webb et al., 2017). Similarly, solid cancers generate local microenvironments with dysregulated pH regardless of the tissue origin or genetic background (Corbet & Feron, 2017; Webb et al., 20110. This is a direct result of the high proliferative and glycolytic rates characteristic of cancer cells, which generate more lactate and protons than normal cells (also known as the Warburg effect) (Warburg, 1956). To maintain a neutral intracellular pH, these cationic species are pumped out of the cells, resulting in a lower extracellular pH compared to non-tumor tissues (Corbet & Feron, 2017). The typical pH of tumor tissues ranges from 6.5-6.9, with values as low 5.85 reported (Wike-Hooley et al., 1984; Johnston et al., 2019), while that of normal-tissue cells is 7.2-7.5 (Webb et al., 2011; Kato et al., 2013). Acidosis seems to occur very early in tumor formation (Damaghi et al., 2013), with recent reports observing low pH proton “halos” surrounding even a single tumor cell (Wei et al., 2019), suggesting pH could be used to target micro-metastases.

The pH difference between normal and cancerous tissues offers a potential strategy to improve antibody specificity for cancerous cells and reduce toxicities towards normal cells. Protein engineering of pH-dependent antigen binding has been reported for an anti-Her2 antibody (Sulea et al., 2020), but paratope engineering is limited to an individual antibody targeting a specific antigen. By contrast, antibody effector functions are highly dependent on interactions between the conserved Fc and immune receptors. Binding of the antibody Fc to FcγRIIIa on NK cells activates antibody-dependent cell-mediated cytotoxicity (ADCC), which is reported to be the major mechanism of action for several FDA-approved monoclonal antibodies (Scott et al., 2012). The Fc-FcγRIIIa binding affinity is known to impact clinical efficacy: individuals expressing the FcγRIIIa V158 allele with high Fc affinity (K_{d}-200-500 nM) exhibit superior responses to antibody therapeutics than those carrying the low affinity F158 allele (K_{d}-850-4500 nM) (Bowles et al., 2006; Forero-Torres et al., 2012). However, clinical results with the recently approved Margetuximab, an anti-Her2 antibody with an Fc domain engineered for stronger FcγRIIIa binding and improved ADCC, revealed more frequent adverse events for patients receiving Margetuximab than Trastuzumab (Mössner et al., 2010). This suggests that Fc variants with higher FcγRIIIa affinity may exacerbate off-target effects unless immune activities are restricted to the tumor microenvironment.

### SUMMARY

To allow for a broadly applicable pH-selective targeting strategy, provided herein are Fc variants with selective ADCC activity in the acidic tumor microenvironment. The human IgG1 Fc domain was engineered to retain physiological FcγRIIIa affinity at the low tumor tissue pH but have weaker affinity at the neutral pH of normal tissue. Since antibody Fab and Fc domains can be combined in a modular fashion, the acid-Fc provided herein may be combined with Fab arms binding any antigen that would benefit from pH-selective targeting.

In one embodiment, provided herein are recombinant polypeptides comprising: (a) a target-binding domain; and (b) a pH-selective mammalian IgG1 Fc domain, wherein the pH-selective IgG1 Fc domain has higher affinity for FcγRIIIa at pH 6.5 than at pH 7.4.

In some aspects, the pH-selective mammalian IgG1 Fc domain selectively binds to FcγRIIIa in an acidic environment. In some aspects, the pH-selective mammalian IgG1 Fc domain has reduced affinity for FcγRIIIa at neutral pH as compared to a wild-type mammalian IgG1 Fc domain. In some aspects, the pH-selective mammalian IgG1 Fc domain has an affinity for FcγRIIIa at neutral pH that is at least about 2-fold, at least about 3-fold, at least about 4-fold, or at least about 5-fold lower than the affinity of a wild-type mammalian IgG1 Fc domain for FcγRIII.

In some aspects, the pH-selective mammalian IgG1 Fc domain has an equivalent affinity for FcγRIIIa at pH 6.5 as compared to a wild-type mammalian IgG1 Fc domain. In some aspects, the pH-selective mammalian IgG1 Fc domain has an affinity for FcγRIIIa at pH 6.5 that is within about 2-fold of the affinity of a wild-type mammalian IgG1 Fc domain for FcγRIIIa at pH 6.5.

In some aspects, the pH-selectivity is determined as the ratio of the affinity for FcγRIIIa at pH 6.5 versus the affinity at pH 7.4. In some aspects, the pH-selective mammalian IgG1 Fc domain has an equivalent affinity for FcRn as a wild-type mammalian IgG1 Fc domain. In some aspects, the pH-selective mammalian IgG1 Fc domain has an affinity for FcRn that is within about 2-fold of the affinity of a wild-type mammalian IgG1 Fc domain for FcRn.

In some aspects, the pH-selective mammalian IgG1 Fc domain has selective ADCC activity in an acidic environment. In some aspects, the pH-selective mammalian IgG1 Fc domain has reduced ADCC activity at pH 7.4 as compared to a wild-type mammalian IgG1 Fc domain. In some aspects, the pH-selective mammalian IgG1 Fc domain has ADCC activity at neutral pH that is at least about 2-fold, at least about 3-fold, at least about 4-fold, at least about 5-fold, at least about 6-fold, at least about 7-fold, at least about 8-fold, at least about 9-fold, or at least about 10-fold lower than the ADCC activity of a wild-type mammalian IgG1 Fc domain.

In some aspects, the pH-selective mammalian IgG1 Fc domain comprises a substitution at the following position(s): S267, H268, Y296, and/or S298, wherein the positions are numbered according to the EU numbering system (which are equivalent to positions 40, 41, 69, and 71 of SEQ ID NO: 1). In some aspects, the pH-selective mammalian IgG1 Fc domain comprises an amino acid sequence of SEQ ID NO: 3.

In some aspects, the pH-selective mammalian IgG1 Fc domain comprises the following substitutions: S267E, H268D, and Y296H, wherein the positions are numbered according to the EU numbering system (which are equivalent to positions 40, 41, and 69 of SEQ ID NO: 1). In some aspects, the pH-selective mammalian IgG1 Fc domain comprises an amino acid sequence of SEQ ID NO: 4.

In some aspects, the pH-selective mammalian IgG1 Fc domain comprises the following substitutions: S267G, H268D, and Y296H, wherein the positions are numbered according to the EU numbering system (which are equivalent to positions 40, 41, and 69 of SEQ ID NO: 1). In some aspects, the pH-selective mammalian IgG1 Fc domain comprises an amino acid sequence of SEQ ID NO: 5.

In some aspects, the pH-selective mammalian IgG1 Fc domain comprises the following substitutions: S267D, H268D, and S298R, wherein the positions are numbered according to the EU numbering system (which are equivalent to positions 40, 41, and 71 of SEQ ID NO: 1). In some aspects, the pH-selective mammalian IgG1 Fc domain comprises an amino acid sequence of SEQ ID NO: 6.

In some aspects, the pH-selective mammalian IgG1 Fc domain comprises an amino acid sequence at least 90% identical to SEQ ID NO: 1 or 2.

In some aspects, the pH-selective mammalian IgG1 Fc domain is glycosylated. In some aspects, the pH-selective mammalian IgG1 Fc domain is glycosylated at residue N297, wherein the position is numbered according to the EU numbering system.

In some aspects, the target-binding domain comprises a peptide that interacts with an antigen of interest. In some aspects, the target-binding domain comprises an antigen-binding portion of an antibody that recognizes an antigen of interest. In some aspects, the target-binding domain comprises an immunoglobulin variable domain. In some aspects, the target-binding domain comprises at least a portion of a ligand that interacts with the antigen of interest. In some aspects, the target-binding domain binds to a tumor antigen or a viral antigen.

In some aspects, the recombinant protein selectively interacts with the target in an acidic microenvironment. In some aspects, the recombinant protein selectively interacts with the target as expressed on tumor cells than as expressed on normal cells.

In some aspects, the recombinant polypeptide is a recombinant antibody or antibody fragment. In some aspects, the antibody fragment is a single chain antibody. In some aspects, the recombinant antibody is a chimeric antibody or bispecific antibody. In some aspects, the recombinant antibody or antibody fragment is a human or humanized antibody or antibody fragment.

In some aspects, the recombinant antibody comprises an immunoglobulin variable domain derived from an antibody that binds a tumor-associated antigen, such as, for example, and anti-Her2 antibody, an anti-CD44 antibody, or an anti-EGFR antibody.

In some aspects, the recombinant antibody comprises an immunoglobulin variable domain derived from an antibody that inhibits an immune checkpoint protein, such as, for example, and anti-PD-L1 antibody, an anti-PD-1 antibody, or an anti-CTLA4 antibody.

In some aspects, the recombinant antibody or antibody fragment is fused to an imaging agent. In some aspects, the recombinant antibody or antibody fragment is labeled. In some aspects, the label is a fluorescent label, an enzymatic label, or a radioactive label. In some aspects, the recombinant antibody or antibody fragment is coupled to a therapeutic, a reporter, or a targeting moiety. In some aspects, the therapeutic is a nucleotide, a peptide, a small molecule, a therapeutic radionuclide, a chemotherapeutic, a tumor suppressor, an apoptosis inducer, an enzyme, a second antibody, an siRNA, a hormone, a prodrug, or an immunostimulant.

In one embodiment, provided herein are isolated nucleic acids encoding a recombinant polypeptide, or a portion of a recombinant polypeptide, provided herein. In some aspects, the isolated nucleic acid is a DNA molecule. In some aspects, the isolated nucleic acid is an RNA molecule. In some aspects, the isolated nucleic acid is an mRNA molecule. In cases wherein the recombinant polypeptide is a heterodimeric molecule, each polypeptide of the heterodimer may be encoded on a separate mRNA molecule. Alternatively, each polypeptide of the heterodimer may be encoded on a single bicistronic mRNA molecule.

In one embodiment, provided herein are expression vectors comprising a nucleic acid sequence encoding a recombinant polypeptide, or a portion of a recombinant polypeptide, provided herein.

In one embodiment, provided herein are hybridomas or engineered cells comprising a nucleic acid encoding a recombinant polypeptide provided herein.

In one embodiment, provided herein are methods of making a recombinant polypeptide as provided herein, the method comprising culturing a hybridoma or engineered cell comprising a nucleic acid encoding the recombinant polypeptide under conditions that allow expression of the recombinant polypeptide and optionally isolating the recombinant polypeptide from the culture.

In one embodiment, provided herein are pharmaceutical formulations comprising one or more recombinant polypeptide as provided herein or one or more mRNA encoding at least one recombinant polypeptide as provided herein.

In one embodiment, provided herein are methods of treating a subject comprising administering an effective amount of the pharmaceutical formulation as provided herein to the subject. In some aspects, the subject has a cancer, such as, for example, a cancer of the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the methods provide for selective targeting of cancer cells expressing the target as compared to targeting of healthy cells expressing the target. In some aspects, the methods further comprise administering at least a second anti-cancer therapy to the subject, such as, for example, a chemotherapy, immunotherapy, radiotherapy, gene therapy, surgery, hormonal therapy, anti-angiogenic therapy, or cytokine therapy.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Display of human IgG1 Fc on the CHO cell surface. (FIG. 1A) Schematic of the Fc CHO display construct and the staining system. The human IgG1 CH2 and CH3 regions were appended with an N-terminal murine IgK secretory leader sequence (LS) and C-terminal (Gly₃Ser)₂ linker (GS) and PDGFR transmembrane domain into the pPyEBV vector. Cells were stained with biotinylated FcgRIIIa (allele V158) monomer followed by streptavidin-PE. To prevent competition between stains, cells were stained separately to monitor Fc display level with anti-human Fc-Alexa Fluor 647 (AF647) to detect display levels. The wildtype Fc (WT) construct, an Fc variant that loses binding to FcgRIIIa (LALAPG), and an Fc variant with enhanced binding to FcgRIIIa (SDALIE) were transfected into CHO cells, stained, and assayed for display levels (FIG. 1B, left plot, with “Untransfected” being the left peak) and binding to FcγRIIIa (FIG. 1B, right plot, with “Untrasnfected” and “LALAPG” being the left peaks, “WT” being the middle peak, and “SDALIE” being the right peak) separately via flow cytometry, with untransfected controls also shown. Data shown are representative of three experimental repeats.

FIGS. 2A-2B. Structural interactions between Fc and FcgRIIIa. (FIG. 2A) The crystal structure of human Fc complexed with FcgRIIIa (PDB 3SGJ) is shown with the two Fc homodimer chains in green (chain A) and blue (chain B), FcgRIIIa in pink and the Fc glycosylations in grey sticks. FcgRIIIa H134, H135 are shown in hot pink spheres. The Fc residues altered in the library (Table 1) are shown in dark green spheres on chain A. Residues L234, L235, G236, G237, S267, A327 were altered to acidic residues since they are within <6.5 Å of FcgRIIIa H134 and H135, while chain A residues E233, Y296, S298 were altered to histidines since they are <5 Å of polar FcgRIIIa residues. The only Fc histidine in the interface, H268, was allowed to be replaced with Y/A/D/S. The corresponding residues on chain B are highlighted in dark blue. (FIG. 2B) The side chain of the existing histidine residues in the Fc-FcγRIIIa interface are shown along with Fc D265 that forms hydrogen bonds with H134. The only FcγRIIIa residue that are in proximity (<5 Å) to Fc H268, K131, is also shown. Molecular graphics and analyses performed with UCSF ChimeraX (Pettersen et al., 2021; Goddard et al., 2018).

FIGS. 3A-3C. Fc variants with pH-dependent binding to FcγRIIIa isolated from CHO display library. (FIG. 3A) The Fc display library was transfected into CHO-T cells and stained for display with anti-human Fc-AF647 (with the left peak being “Untransfected”, the middle peak being “Library”, and the right peaks being “WT” and “LALAPG”) and binding to FcγRIIIa-SA-PE (with the left peaks being “Untransfected” and “LALAPG”, the middle peak being “Library”, and the right peak being “WT”). (FIG. 3B) The library was sorted over two rounds for binding to FcγRIIIa-SA-PE at pH 6.5, followed by two rounds of enrichment for stronger pH-dependence. To select for pH-dependent binding, the library was stained first with 50 nM FcgRIIIa-SA-AF647 at pH 7.4, washed, then stained with 20 nM FcγRIIIa-SA-PE at pH 6.5. The gate shown is representative of the sorting gate used in round four. (FIG. 3C) Individual clones selected during rounds three and round four of the library were isolated and sequenced before being transfected into fresh CHO-T cells, stained, and assessed for Fc variants display and binding to FcγRIIIa at pH 6.5 and pH 7.4 via flow cytometry. Selectivity for each variant was calculated as the ratio of the percentage of cells binding FcγRIIIa at pH 6.5 divided by that at pH 7.4. Data shown are pooled from three experimental replicates.

FIGS. 4A-4C. Binding of selected and engineered Fc variants to FcgRIIIa at pH 6.5 and 7.5. Selected Fc variants with the native E233, L234 and L235 residues were expressed as IgG proteins with hu4D5 Fab and evaluated for binding to purified human FcγRIIIa. (FIG. 4A) ELISA was performed with antibody coated at 2 μg/ml, followed by FcγRIIIa V158 and detected with anti-His-HRP with all incubation and wash buffers maintained at the indicated pH. For the pH 7.4 graph, the lines are, from left to right at A450 of 0.5 nm, “Wild-type Fc”, “3F2”, “Acid-Fc”, and “3E2”. For wild-type and the acid-Fc variants, BLI was performed on an OctRed96 with serially diluted FcγRIIIa V158 (63-2000 nM) and F158 (156-5000 nM) binding to antibodies captured on FAB2G tips. (FIG. 4B) Initial kinetic response was fitted to a 1:1 model. (FIG. 4C) Equilibrium response was fitted to Langmuir isotherm. In each graph, the top line is “Wild-type Fc”. Obtained K_{d} from kinetic and steady state analysis are shown in Table 2. Data representative of four replicates.

FIGS. 5A-5B. Clearance rates for hu4D5-Fc variant antibodies in transgenic mice. (FIG. 5A) Homozygous Tg32 mice (n=4) expressing the human FcRn under the human promoter were administered 2 mg/kg of each antibody intra-peritoneally and tail vein samples were collected every ˜3 days. Serum concentrations of the administered human antibody were determined by antigen-specific ELISA and plotted against time to determine the beta elimination half-life. (FIG. 5B) Serum elimination half-lives of the modified-Fc 4D5 variants.

FIGS. 6A-6C. In vitro cellular cytotoxicity mediated by hu4D5-Fc variant antibodies. (FIG. 6A) Flow cytometric ADCP assay with THP-1 monocytic cells. Her2-coated fluorescent beads that are also labeled with an intracellular pH fluorogenic probe were incubated with THP-1 cells and hu4D5 antibodies with wild-type Fc, acid-Fc, or an isotype antibody that does not bind to Her2. After incubation, cells were scanned by flow and phagocytosis score was calculated as (% beads association+% bead internalization)*GMFI(beads association). (FIG. 6B) Antibody variants (50 ng/mL) were added to calcein-loaded SKOV3 target cells in the presence of NK92 cells stably expressing FcγRIIIa V158. Cells and antibodies were incubated together for four hours at E:T ratio of 10:1. Data shown are representative of replicate experiments. (FIG. 6C) Dose-dependent ADCC assay of hu4D5 antibodies with acid-Fc or wild-type Fc against calcein-loaded SKBR3 target cells in the presence of NK92(V158) cells. Data shown are pooled from two experimental replicates, each performed with two technical replicates, with the mean and standard deviation of pooled data shown. *, p<0.05; **, p<0.01; ***, p<0.001 determined by t-test in GraphPad.

FIGS. 7A-7B. Contributions of acid-Fc residue changes to pH selectivity. Individual changes of acid-Fc were expressed in Fc on the surface of CHO cells and stained for display levels and binding to FcγRIIIa via flow cytometry. Ratio of binding at pH 6.5 to binding at pH 7.4 for these variants is shown in (FIG. 7A), data represents mean and range from two experimental repeats. * p<0.05. (FIG. 7B) Presumptive interactions of acid-Fc mutations that mediate pH selectivity. Structure from PDB 3SGJ with residues changed to the identity in acid-Fc using the most common rotamer in ChimeraX. Under acidic conditions, S267E may form electrostatic interactions with protonated H134 on FcγRIIIa, while H268D may form a salt bridge with K131 of FcγRIIIa.

FIG. 8. Schematic of FACS selection staining strategy. Cells were labeled with 50 nM of AF647-labeled monomeric FcgRIIIa at pH 7.4, and then washed with flow buffer at pH 7.4 to allow clones binding weakly at neutral pH to dissociate. The cells were then stained with PE-labeled monomeric FcgRIIIa at pH 6.5, washed with flow buffer at pH 6.5 and sorted by FACS to collect clones with high PE and low AF647 fluorescence that preferentially bind at low pH.

FIGS. 9A-9D. Characterization of initially selected Fc variants. (FIG. 9A) When transfected into CHO-T cells and analyzed as clonal populations displaying a single Fc variant, similar Fc expression levels are observed by flow cytometry using anti-human-Fc AF647 antibodies. (FIG. 9B) While wild-type Fc showed similar binding levels to FcγRIIIa at pH 6.5 and pH 7.4, binding for all variants at pH 7.4 was reduced compared to binding at pH 6.5. (FIG. 9C) Full-length antibodies with hu4D5 Fab arms and Fc variants (2 μg/mL) were immobilized on ELISA plates and binding to His-tagged monomeric FcγRIIIa detected by anti-His HRP by ELISA. (FIG. 9D) All variants showed greatly reduced FcγRIIIA binding versus wild-type at both pH values.

FIGS. 10A-10B. Characterization of initially selected Fc variants. (FIG. 10A) Biophysical characterizations of hu4D5 antibody variants by SDS-PAGE gel. (FIG. 10B) Production of recombinant FcgRs. Proteins were transiently expressed in Expi293 cells and purified via c-terminal His tags.

FIGS. 11A-11B. BLI was performed with serially diluted antibodies (62.5 nM to 2 μM) binding to biotinylated FcgRIIIa V158 and F158 captured on streptavidin tips (FIGS. 4B, 4C). Equilibrium response was fitted to Langmuir isotherm for equilibrium K_{d} analysis. In FIG. 11B, the pH 6.5 graph, the lines represent, from top to bottom at a concentration of 1000 nM, “WT”, “acid-Fc”, “3E2”, and “3F2”. In FIG. 11B, the pH 7.4 graph, the top line is “WT”.

FIGS. 12A-12C. BLI traces for hu4D5 antibody variants binding to (FIG. 12A) FcgRIIa R131, (FIG. 12B) FcgRIIa H131, and (FIG. 12C) FcgRIIb at pH 6.5 and pH 7.4. Antibody variants were captured on FAB2G sensors, association and dissociation rates were measured with serially diluted FcgRs. Equilibrium response was fitted to Langmuir isotherm for equilibrium K_{d} analysis (Table 2).

FIGS. 13A-13B. Biophysical characterizations of hu4D5 antibody variants. (FIG. 13A) SEC traces by Superdex S200 column with an Akta FPLC. (FIG. 13B) Antibody variants (100 μg/mL) were mixed with Protein Thermal Shift™ (Thermal Fisher) dye, and the melt curve with ramp rate of 0.05° C./see was measured by real-time PCR using ViiA7™ machine.

FIG. 14. ELISA of FcRn binding to immobilized antibody comprised of hu4D5 Fab arms and the indicated Fc variants at pH 6.0 and pH 7.4. Purified antibodies were immobilized at 2 g/ml followed by blocking, titration of FLAG-tagged FcRn in buffer at the indicated pH and detection with anti-FLAG HRP. In the pH 6.0 graph, the left-shifted line represents “YTE”.

FIG. 15. Characterization of single residue Fc variants. Single residue variants were expressed on the CHO cell surface and assessed for FcγRIIIa binding at both pH values by flow cytometry. In each pair of columns, the left column is pH 6.5 and the right column is pH 7.4.

### DETAILED DESCRIPTION

Despite the exquisite specificity and high affinity of antibody-based cancer therapies, side effects can occur since the tumor-associated antigens targeted are often also present on healthy cells. However, the low pH of the tumor microenvironment provides an opportunity to develop conditionally active antibodies with enhanced tumor specificity. Accordingly, the human IgG1 Fc domain was engineered for pH-selective FcγRIIIa binding and antibody-dependent cellular cytotoxicity (ADCC). The Fc was displayed on the surface of mammalian cells and a site-directed library was generated by altering Fc residues in the Fc-FcγRIIIa interface to support interactions with positively charged histidines. A competitive staining strategy and flow cytometric selection were used to isolate Fc variants exhibiting reduced affinities at neutral pH but physiological affinities at the tumor-typical pH 6.5. Antibodies comprised of anti-Her2 Fab arms and acid-Fc exhibited a 23-fold increased pH selectivity for FcγRIIIa versus wild-type Fc, based on the ratio of equilibrium binding constants, K_{d,7.4}/K_{d,6.5}. This variant retained physiological binding to FcRn and FcγRIIa H131, with enhanced binding to FcγRIIb and FcγRIIa R131 and unmodified pharmacokinetics in transgenic mice expressing human FcRn. In vitro ADCC assays with human NK92 effector and Her2-positive target cells demonstrated similar activities for both Fes at pH 6.5 but ˜19-fold reduced ADCC for acid-Fc versus WT Fc at pH 7.4. Acid-Fc provides for pH-selective Fc activation allowing for two dimensions of selective tumor cell targeting.

### I. ASPECTS OF THE PRESENT DISCLOSURE

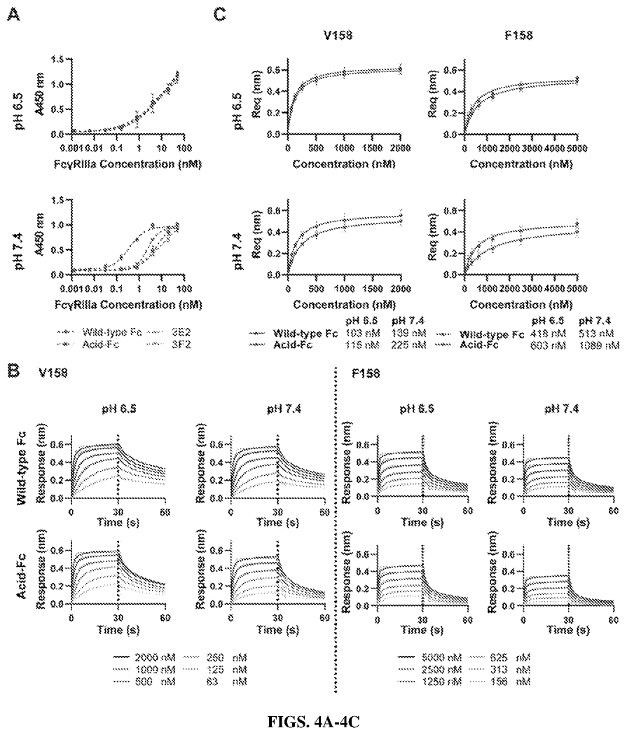

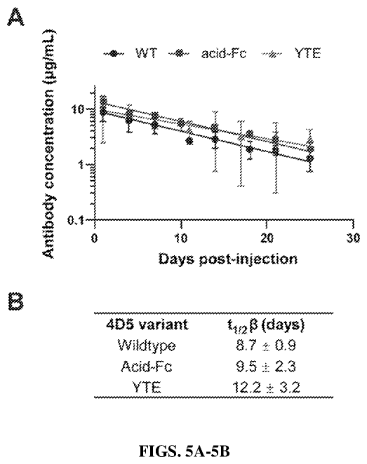

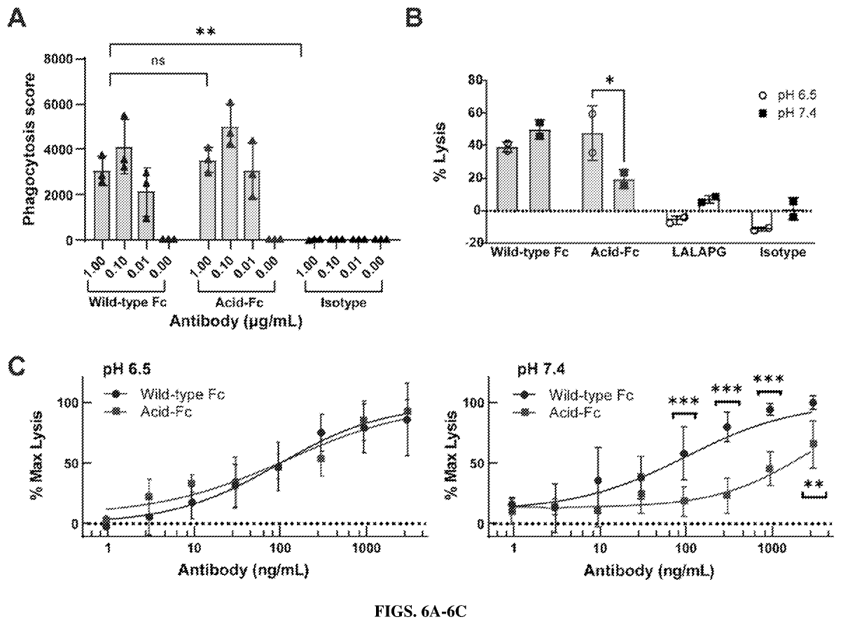

Mammalian cell display was used to identify human IgG1 Fc variants with pH-selective binding to FcγRIIIa and activation of ADCC. Acid-Fc contains three residue changes that minimally impact FcγRIIIa affinity at pH 6.5 but reduce it ˜3-fold at pH 7.4, whereas the wild-type Fc shows minimal pH selectivity (FIG. 4, Table 2). While these affinities were measured with purified proteins binding in a 1:1 stoichiometry, the physiologically relevant interaction involves ˜10⁵ FcγRIIIa receptors expressed on an NK cell (Hatjiharissi et al., 2007), which trigger signaling after binding clustered antibody Fc domains due to their Fab arms recognizing multiple ligand molecules on the target cell surface (Nimmerjahn & Ravetch, 2008). Since this is a complex and multivalent binding interaction, cellular effects are difficult to extrapolate from measurements with purified proteins. To assess the biological activity of acid-Fc, in vitro ADCC assays with human NK-92 effector cells, anti-Her2 antibodies, and Her2-positive cells were used (FIGS. 6B,6C). Whereas the wild-type Fc exhibited minimal pH-selectivity, acid-Fc demonstrated ˜19-fold weaker activity at pH 7.4 than pH 6.5 without reducing binding to other Fcγ receptors or pharmacokinetics in Tg32 mice (FIG. 5).

A mammalian display platform was used to enable the screening of Fc variants in the presence of native glycan. This is an advantage not shared by yeast and bacterial display systems and one of the reasons that many prior Fc engineering efforts employed screening of individual point variants (Mimoto et al., 2013) and computational design strategies (Lazar et al., 2006). The presence of the native sugar during high-throughput selection is especially important for Fc engineering because carbohydrate moieties occupy ˜21% (261 Å²) of the total Fc-FcγRIIIa interface area (Mizushima et al., 2011). Glycosylation at residue N297 stabilizes the Fc region in an “open” conformation, which is critical for binding to and activating classical Fc receptors on immune cells (Liu et al., 2020). Selection of variants in the presence of different glycosylation profiles, e.g., the hyper-glycosylation provided by yeast, may not be predictive of final antibody characteristics when expressed in mammalian cells, especially when sugar-proximal residues are altered. Fc variants that recapitulate this open state in the absence of glycosylation have been identified that are compatible with yeast and bacterial display (Sazinsky et al., 2008; Jung et al., 2013; Lee et al., 2017; Kang et al., 2019) but this imposes additional constraints on the variants selected.

Mammalian display systems for antibody engineering have been reported. A lentiviral-based mammalian display platform was recently reported by Chen et al. (2021) who screened >10⁴ Fc variants in HEK293T cells to identify Fc variants with enhanced FcγRIIIa (˜10-fold improved K_{d}) and FcγRIIb binding (˜2.6-fold improved K_{d}) and enhanced cellular activities. In the studies provided herein, diversity of >10⁶ was achieved without employing lentivirus. CHO cell display has the additional advantage that selected proteins are expected to be compatible with existing large-scale manufacturing processes since CHO cells are used to manufacture most protein therapeutics (Jayapal et al., 2007). Future efforts will aim to further reduce the display level to mitigate the impact of avidity during selection, as well as to restrict the flexibility of the hinge region by expression of a single-chain variable fragment-Fc fusion.

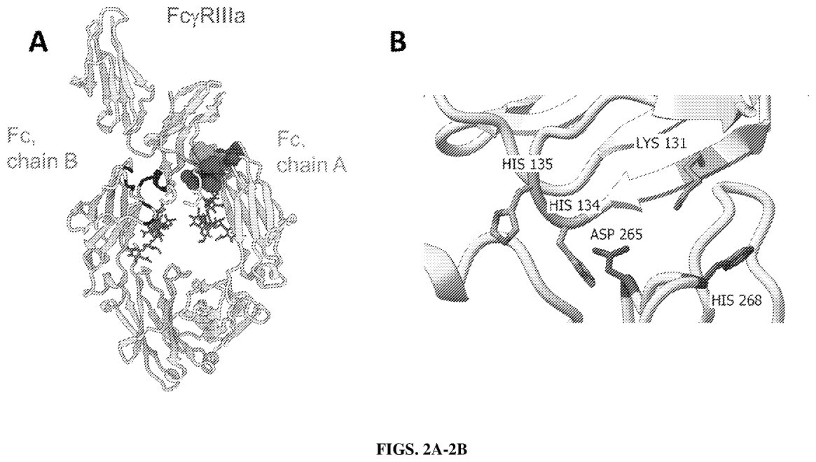

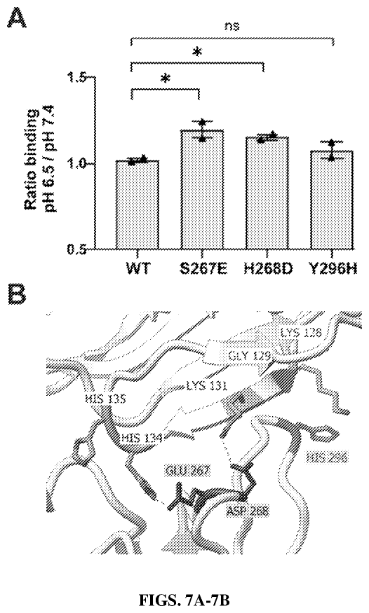

Fc changes were identified at four positions in variants exhibiting pH-selective activity, with the S267E, H268D and Y296H changes present in acid-Fc (Table 1). Crystal structures show an asymmetric Fc-FcγRIIIa binding interface dominated by van der Waals contacts and several hydrogen bonds (Sondermann et al., 2000; Mizushima et al., 2011), with FcγRIIIa residues H134 and H135 mediating multiple non-covalent interactions (FIG. 2B). Notably, all Fc variants included changes at position S267. An S267A substitution was previously shown to have no impact on FcγRIIIa binding (Shields et al., 2001), suggesting the selected S267G change is also inert. By contrast, the S267E and S267D changes could form electrostatic interactions with protonated H134 and H135 residues on FcγRIIIa (FIG. 7). An H268D change was also observed in all variants and previously reported to directly support electrostatic FcγRIIIa interactions and indirectly influence side chain conformations of adjacent Fc residues (Mimoto et al., 2013). When this native Fc histidine is protonated at low pH, H268 may reduce FcγRIIIa binding by charge-charge repulsion, but replacement with a negatively charged aspartic acid may support electrostatic interactions with FcγRIIIa K131 at pH 6.5 and 7.4 (FIG. 7). In all variants except the 3E lineage, a new histidine was introduced at position Y296. This residue is adjacent to the N297 glycosylation and is reported to interact with K128 and G129 on FcγRIIIa, as well as sugars on FcγRIIIa and Fc (Mizushima et al., 2011) but does not appear to mediate pH-selective interactions. Analysis of single residue variants is consistent with these interpretations (FIG. 7). Without being bound by theory, this structural analysis provides a rationale to explain the pH-selective Fc-FcγRIIIa binding observed for these variants.

Effector cell activation induced by FcγRIIIa requires the high-avidity crosslinking of antibody-coated target cells with effector cells because of the weak Fc-FcγRIIIa affinity (˜200-400, or 850-4500 nM, for the V158 and F158 alleles, respectively) (Mössner et al., 2010; Ahmed et al., 2016). As a result, cellular assays are more physiologically relevant than affinities measured with soluble proteins and modest changes in FcγRIIIa binding affinity can result in larger increases in efficacy. For example, an Fc variant with ˜10-fold tighter K_{d} led to ˜100-fold more sensitive ADCC (Stavenhagen et al., 2007). As such, in vitro ADCC assays were performed using human NK92 cells stably expressing the high-affinity V158 FcγRIIIa allele in the presence of cell lines with high or medium Her2 expression levels to mimic clinical variation (˜10⁶/cell for SKBR3 and ˜10⁵/cell for SKOV3 cells (Lazar et al., 2006)). This resulted in ˜2.4-fold difference when using one antibody concentration and SKOV3 cells (FIG. 6B) and a ˜19-fold pH selectivity based on analysis of the full antibody dose-response curves with SKBR3 cells (FIG. 6C).

FcγRIIIa binding and ADCC are critical for the success of tumor immunotherapies (Scott et al., 2012) and mediate many “on target, off tumor” effects. However, it is important to understand whether the acid-Fc changes may also impact complement and functions mediated by other FcγRs, such as inhibitory activities and antibody-dependent phagocytosis, as these can also contribute to protection (Kang et al., 2019). For example, the S267E change was previously shown to enhance complement-dependent cytotoxicity by 3-fold due to increased C1q binding (Moore et al., 2010). Similarly, while acid-Fc did not exhibit pH-selective binding to the highly similar FcγRIIa and IIb receptors (˜94% sequence identity), likely because the FcγRIIIa H134 and H135 histidines are not conserved, the affinity of acid-Fc for two of these receptors increased >10-fold versus wild-type Fc (Table 5). Residues R134 and R131 on FcγRIIb and FcγRIIa, respectively, are structurally homologous to H134 on FcγRIIIa. Based on the structural analysis above, and without being bound by theory, the S267E and H268D changes in acid-Fc may form favorable charge-charge interactions with these positively charged H134 homologs. This is supported by data showing that acid-Fc binds FcγRIIb and the FcγRIIa R131 allele with similar affinity increases, while a slight increase in binding to the H131 allele only at pH 6.5, when this residue is expected to be protonated, was observed. Since FcγRIIb is not expressed on NK cells (Nimmerjahn & Ravetch, 2008), this is not expected to impact ADCC activity. No significant difference was observed for phagocytic activities of wild-type and acid-Fc using monocytic THP-1 cells (FIG. 6A).

Site-specific drug delivery strategies based on nano-scale carriers responding to acidic pH, such as peptides, liposomes, micelles, polymeric nanoparticles and polymersomes have been reported (Corbet & Feron, 2017; Andreev et al., 2010), but expanding these approaches to protein therapeutics is a new concept. The premise that pH-selective proteins can selectively target tumors in vivo was first reported for the endogenous immune checkpoint molecule V-domain immunoglobulin suppressor of T cell activation (VISTA), which is rich in histidine residues and suppresses immune responses by binding the P-selectin glycoprotein ligand-1 to trigger immune-inhibitory signals only at low pH (Johnston et al., 2019). Monoclonal antibodies preferentially binding VISTA at low pH, but not non-pH-selective antibodies, accumulated in tumors of mice expressing human VISTA, providing in vivo evidence for the concept of pH-selective tumor targeting by proteins. Engineering to increase antibody affinity for a tumor-associated antigen at low but not neutral pH was reported in greater detail by Sulea et al. (2020) This work used structure-based computational histidine mutagenesis to guide engineering of the low affinity Her2-binding antibody bH1. Antibody variants increased pH selectivity from 0.23 for bH1 to 5.8 as measured pH 7.4/pH 5.0 K_{d} ratios, with the drawback that the most selective variants attained only a modest 50 nM Her2 affinity at pH 5.0, versus 13 nM for bH1. In the studies provided herein, similar K_{d} selectivity and ADCC ratios were achieved in an antigen-agnostic manner by modifying the Fc domain.

Antibodies with increased tumor selectivity have the potential to mitigate the “on-target off-tumor” side effects and target-mediated deposition common to many antibody therapeutics. A shared characteristic that distinguishes many tumor types from healthy tissues is tumor acidity (Corbet & Feron, 2017; Webb et al., 2011; Damaghi et al., 2013; Wei et al., 2019), suggesting antibodies with pH-selective activity, such as acid-Fc, may provide a secondary means of selective antibody activation. Future in vitro experiments with target cell lines ranging in Her2 expression levels and primary human effector cells will be performed to determine conditions resulting in the greatest differential ADCC and the impact of selected Fc changes on complement and antibody-dependent cellular phagocytosis. Mapping of tumor acidosis in vivo in humans and mice supports the presence of appropriate pH values for even small metastases (Wei et al., 2019; Ferrauto et al., 2014). Overall, conditionally active Fc domains provide a general strategy to reduce antibody off-tumor effects.

One class of antibody therapeutics that can benefit from a conditionally active Fc domain are antagonists of immune inhibitory molecules. Known inhibitors of immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, HLA-DRB1, HLA-DQA1, HLA-E, killer-cell immunoglobulin (KIR), lymphocyte activation gene-3 (LAG-3, also known as CD223), Mer tyrosine kinase (MerTK), NKG7, programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, immune checkpoint inhibitors targeting the PD-1 axis and/or CTLA-4 have received FDA approval broadly across diverse cancer types.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10DI, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted for immunomodulation is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGDO13 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted for immunomodulation is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted for immunomodulation is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted for immunomodulation is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

### II. DEFINITIONS

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

Other terms used in the fields of recombinant nucleic acid technology, microbiology, immunology, antibody engineering, and molecular and cell biology as used herein will be generally understood by one of ordinary skill in the applicable arts.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### III. ANTIBODIES AND MODIFICATIONS OF ANTIBODIES

Provided herein are antibodies and antibody fragments having modified Fc domains that selectively bind to FcγRIIIa in an acidic environment. The pH-selective mammalian IgG1 Fc domains may have reduced affinity for FcγRIIIa at neutral pH as compared to a wild-type mammalian IgG1 Fc domain while having equivalent affinity for FcγRIIIa at pH 6.5 as compared to a wild-type mammalian IgG1 Fc domain. Such antibodies and antibody fragments may be produced using methods described herein.

Provided herein are antibodies or antibody fragments comprising a human IgG1 heavy chain Fc domain comprising an engineered version of the following amino acid sequence:

(SEQ ID NO: 1)

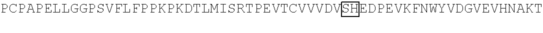

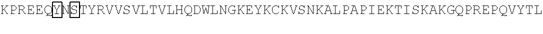

PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK.

In some aspects, the Fc domain is modified at amino acid Ser 267 (boxed in SEQ ID NO: 1 in the preceding paragraph) to alter Fc receptor interactions in a pH-dependent manner, for example Ser267Glu (S267E) or Ser267Asp (L267D) or Ser267Gly (S267G). In some aspects, the Fc domain is modified at amino acid His268 (boxed in SEQ ID NO: 1 in the preceding paragraph) to alter Fc receptor interactions in a pH-dependent manner, e.g., His268Asp (H268D). In some aspects, the Fc domain is modified at amino acid Tyr296 (boxed in SEQ ID NO: 1 in the preceding paragraph), e.g., Tyr296His (Y296H). In some aspects, the Fc domain is altered at amino acid Ser298 (boxed in SEQ ID NO: 1 in the preceding paragraph), e.g., Ser298Arg (S298R). All residue numbers are according to EU numbering (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

Provided herein are antibodies or antibody fragments comprising a human IgG1 heavy chain Fc domain comprising an engineered version of the following amino acid sequence:

(SEQ ID NO: 2)

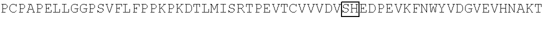

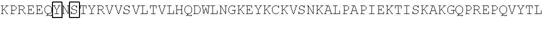

PPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK.

In some aspects, the Fc domain is modified at amino acid Ser 267 (boxed in SEQ ID NO: 1 in the preceding paragraph) to alter Fc receptor interactions in a pH-dependent manner, for example Ser267Glu (S267E) or Ser267Asp (L267D) or Ser267Gly (S267G). In some aspects, the Fc domain is modified at amino acid His268 (boxed in SEQ ID NO: 1 in the preceding paragraph) to alter Fc receptor interactions in a pH-dependent manner, e.g., His268Asp (H268D). In some aspects, the Fc domain is modified at amino acid Tyr296 (boxed in SEQ ID NO: 1 in the preceding paragraph), e.g., Tyr296His (Y296H). In some aspects, the Fc domain is altered at amino acid Ser298 (boxed in SEQ ID NO: 1 in the preceding paragraph), e.g., Ser298Arg (S298R). All residue numbers are according to EU numbering (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

Provided herein are antibodies or antibody fragments comprising a human IgG1 heavy chain Fc domain comprising the following amino acid sequence:

(SEQ ID NO: 3)

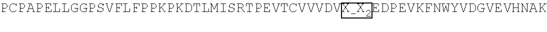

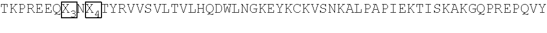

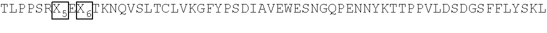

TVDKSRWQQGNVFSCSVMHEALHNHYTQKSLSLSPGK,

wherein X₁ is Ser, Glu, Gly, or Asp; X₂ is His or Asp; X₃ is Tyr or His; X₄ is Ser or Arg; X₅ is Asp or Glu; and X₆ is Leu or Met.

Provided herein are antibodies or antibody fragments comprising a human IgG1 heavy chain Fc domain comprising the following amino acid sequence:

(SEQ ID NO: 4)
PCPAPELLGGPSVFLFPPKPKDTLMISRTPEVTCVVVDVEDEDPEVKFNWYVDGVEVHNAKT
KPREEQHNSTYRVVSVLTVLHQDWLNGKEYKCKVSNKALPAPIEKTISKAKGQPREPQVYTL

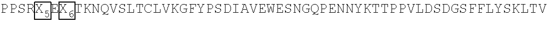

DKSRWQQGNVFSCSVMHEALHNHYTQKSLSLSPGK,

wherein X₅ is Asp or Glu; and X₆ is Leu or Met

Provided herein are antibodies or antibody fragments comprising a human IgG1 heavy chain Fc domain comprising the following amino acid sequence:

(SEQ ID NO: 5)
PCPAPELLGGPSVFLFPPKPKDTLMISRTPEVTCVVVDVGDEDPEVKFNWYVDGVEVHNAKT
KPREEQDNSTYRVVSVLTVLHQDWLNGKEYKCKVSNKALPAPIEKTISKAKGQPREPQVYTL

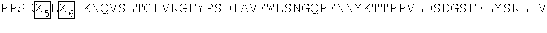

DKSRWQQGNVFSCSVMHEALHNHYTQKSLSLSSPGK,

wherein X₅ is Asp or Glu; and X₆ is Leu or Met.

Provided herein are antibodies or antibody fragments comprising a human IgG1 heavy chain Fc domain comprising the following amino acid sequence:

(SEQ ID NO: 6)
PCPAPELLGGPSVFLFPPKPKDTLMISRTPEVTCVVVDVDDEEDPEVKFNWYVDGVEVHNAKT
KPREEQYNRTYRVVSVLTVLHQDWLNGKEYKCKVSNKALPAPIEKTISKAKGQPREPQVYTL

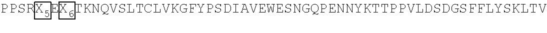

DKSRWQQGNVFSCSVMHEALHNHYTQKSLSLSPGK,

wherein X₅ is Asp or Glu; and X₆ is Leu or Met.

In some aspects, the constant region of the heavy chain of the antibody or antibody fragment is a human IgG1 isotype, having an amino acid sequence:

(SEQ ID NO: 7)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF

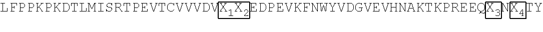

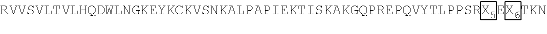

QVSLTCLVKGFYPSDIAVEWESNGQPENNYKTTPPVLDSDGSFFLYSKLTVDKSRWQQGNVF
SCSVMHEALHNHYTQKSLSLSPGK,

wherein X₁ is Ser, Glu, Gly, or Asp; X₂ is His or Asp; X₃ is Tyr or His; X₄ is Ser or Arg; X₅ is Asp or Glu; and X₆ is Leu or Met.

In some aspects, the human IgG1 constant region is modified to comprise either a “knob” mutation, e.g., T366Y, or a “hole” mutation, e.g., Y407T, for heterodimerization with a second constant region (residue numbers according to EU numbering (Kabat, E. A., et al., supra)).

In some aspects, the constant region of the heavy chain of the antibody is a human IgG1 isotype, e.g., an allotype of the human IgG1 isotype, e.g., the IgG1 Glm3 allotype. Exemplary human IgG1 allotypes are described in Magdelaine-Beuzelin et al. (2009) PHARMACOGENET. GENOMICS 19(5):383-7.

In some aspects, the human IgG constant region is modified to enhance FcRn binding. Examples of Fc mutations that enhance binding to FcRn are Met252Tyr, Ser254Thr, Thr256Glu (M252Y, S254T, T256E, respectively) (Dall'Acqua et al. (2006) J. BIOL. CHEM. 281(33): 23514-23524), or Met428Leu and Asn434Ser (M428L, N434S) (Zalevsky et al. (2010) NATURE BIOTECH. 28(2): 157-159). All residue numbers are according to EU numbering (Kabat, E. A., et al., supra).

In some aspects, the human IgG constant region is modified to alter antibody-dependent cellular cytotoxicity (ADCC) and/or complement-dependent cytotoxicity (CDC), e.g., the amino acid modifications described in Natsume et al. (2008) CANCER RES. 68(10): 3863-72; Idusogie et al. (2001) J. IMMUNOL. 166(4): 2571-5; Moore et al. (2010) MABS 2(2): 181-189; Lazar et al. (2006) PROC. NATL. ACAD. SCI. USA 103(11): 4005-4010, Shields et al. (2001) J. BIOL. CHEM. 276(9): 6591-6604; Stavenhagen et al. (2007) CANCER RES. 67(18): 8882-8890; Stavenhagen et al. (2008) ADVAN. ENZYME REGUL. 48: 152-164; Alegre et al. (1992) J. IMMUNOL. 148: 3461-3468.

In some aspects, the human IgG constant region is modified to induce heterodimerization. For example, a heavy chain having an amino acid modification within the CH3 domain at Thr366, e.g., a substitution with a more bulky amino acid, e.g., Tyr (T366W), is able to preferentially pair with a second heavy chain having a CH3 domain having amino acid modifications to less bulky amino acids at positions Thr366, Leu368, and Tyr407, e.g., Ser, Ala and Val, respectively (T366S/L368A/Y407V). Heterodimerization via CH3 modifications can be further stabilized by the introduction of a disulfide bond, for example by changing Ser354 to Cys (S354C) and Y349 to Cys (Y349C) on opposite CH3 domains (see, Carter (2001) J. IMMUNOL. METHODS 248: 7-15).

In some aspects, the constant region of the light chain of the antibody is a human kappa constant region, e.g., a human kappa constant region having the amino acid sequence:

(SEQ ID NO: 8)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC,

In some aspects, the constant region of the light chain of the antibody is a human kappa constant region, e.g., a human kappa constant region having the amino acid sequence:

(SEQ ID NO: 9)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In some aspects, the constant region of the light chain of the antibody is a human lambda constant region, e.g., a human lambda constant region having the amino acid sequence:

(SEQ ID NO: 10)
GQPKANPTVTLFPPSSEELQANKATLVCLISDFYPGAVTVAWKADGSPV
KAGVETTKPSKQSNNKYAASSYLSLTPEQWKSHRSYSCQVTHEGSTVEK
TVAPTEC.

The antibodies and antibody fragments of the present invention have several applications, include the diagnosis and treatment of diseases. As such, antibodies or antibody fragments may be linked diagnostic or therapeutic agents or used without additional agents being attached thereto. The antibodies or antibody fragments may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv, Fd, Fd′, single chain antibody (ScFv), diabody, linear antibody), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

An “isolated antibody” is an antibody that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular instances, the antibody is purified: (1) to greater than 95% by weight of antibody as determined by the Lowry method, and most particularly more than 99% by weight; or (2) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, an isolated antibody will be prepared by at least one purification step.

The basic four-chain antibody unit is a heterotetrameric glycoprotein composed of two identical light (L) chains and two identical heavy (H) chains. The term “heavy chain” as used herein refers to the larger immunoglobulin subunit which associates, through its amino terminal region, with the immunoglobulin light chain. The heavy chain comprises a variable region (V_{H}) and a constant region (C_{H}). The constant region further comprises the C_{H}1, hinge, C_{H}2, and C_{H}3 domains. In the case of IgE, IgM, and IgY, the heavy chain comprises a C_{H}4 domain but does not have a hinge domain. Those skilled in the art will appreciate that heavy chains are classified as gamma, mu, alpha, delta, or epsilon (γ, μ, α, δ, ε), with some subclasses among them (e.g., γ1-γ4, α1-α2). It is the nature of this chain that determines the “class” of the antibody as IgG, IgM, IgA IgD, or IgE, respectively. The immunoglobulin subclasses (isotypes), e.g., IgG1, IgG2, IgG3, IgG4, IgA1, etc. are well characterized and are known to confer functional specialization.

The term “light chain” as used herein refers to the smaller immunoglobulin subunit which associates with the amino terminal region of a heavy chain. As with a heavy chain, a light chain comprises a variable region (V_{L}) and a constant region (C_{L}). Light chains are classified as either kappa or lambda (κ, λ) based on the amino acid sequences of their constant domains (C_{L}). A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

A “variable region” of an antibody refers to the variable region of the antibody light chain or the variable region of the antibody heavy chain, either alone or in combination. The term “variable” refers to the fact that certain segments of the variable regions differ extensively in sequence among antibodies. The variable regions of both the light (V_{L}) and heavy (V_{H}) chain portions mediate antigen binding and define the specificity of a particular antibody for its particular antigen. However, the variability is not evenly distributed across the entirety of the variable regions. Instead, the variable regions consist of relatively invariant stretches called framework regions (FRs) separated by shorter regions of extreme variability called complementarity determining regions (CDRs) or hypervariable regions. The variable regions of native heavy and light chains each comprise four FRs, largely adopting a beta-sheet configuration, connected by three CDRs, which form loops connecting, and in some cases forming part of, the beta-sheet structure. The CDRs complement an antigen's shape and determine the antibody's affinity and specificity for the antigen. There are six CDRs in both V_{L} and V_{H}. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al, Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)). As used herein, a CDR may refer to CDRs defined by any of these numbering approaches or by a combination of approaches or by other desirable approaches. In addition, a new definition of highly conserved core, boundary and hyper-variable regions can be used.

A “constant region” of an antibody refers to the constant region of the antibody light chain or the constant region of the antibody heavy chain, either alone or in combination. The constant regions of the light chain (C_{L}) and the heavy chain (C_{H}1, C_{H}2 or C_{H}3, or C_{H}4 in the case of IgM and IgE) confer important biological properties such as secretion, transplacental mobility, Fc receptor binding, complement binding, and the like. By convention the numbering of the constant region domains increases as they become more distal from the antigen binding site or amino-terminus of the antibody. The constant regions are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The antibody may be an antibody fragment. “Antibody fragments” comprise only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H1} domains; (ii) the Fab′ fragment, which is a Fab fragment having one or more cysteine residues at the C-terminus of the C_{H1} domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single antibody; (vi) the dAb fragment which consists of a V_{H} domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}—C_{H}1-V_{H}-C_{H}1) which, together with complementary light chain polypeptides, form a pair of antigen binding regions.

The antibody may be a chimeric antibody. “Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. For example, a chimeric antibody may be an antibody comprising antigen binding sequences from a non-human donor grafted to a heterologous non-human, human, or humanized sequence (e.g., framework and/or constant domain sequences). Typically, in these chimeric antibodies, the variable region of both light and heavy chains mimics the variable regions of antibodies derived from one species of mammals, while the constant portions are homologous to the sequences in antibodies derived from another. For example, methods have been developed to replace light and heavy chain constant domains of a monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

### A. Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The monoclonal antibodies may also be isolated from phage antibody libraries using the techniques described in Clackson et al., Nature, 352:624-628 (1991) and Marks et al., J. Mol. Biol., 222:581-597 (1991), for example.

Methods for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024, each incorporated herein by reference.

### B. Bispecific and Multispecific Antibodies

Antibodies may be bispecific or multispecific. “Bispecific antibodies” are antibodies that have binding specificities for at least two different epitopes. Exemplary bispecific antibodies may bind to two different epitopes of a single antigen. Other such antibodies may combine a first antigen binding site with a binding site for a second antigen. Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess an antigen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). Taki et al. (2015) describes a bispecific anti-HSP70/anti-CD3 antibody.

Methods for making bispecific antibodies are known in the art. Traditional production of full-length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low.

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

The bispecific antibodies may be composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998)). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and Vu domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

A bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400). Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). The antibodies may also involve sequences or moieties that permit dimerization or multimerization of the receptors. Such sequences include those derived from IgA, which permit formation of multimers in conjunction with the J-chain. Another multimerization domain is the Gal4 dimerization domain.

A multivalent antibody may be internalized (and/or catabolized) faster than a bivalent antibody by a cell expressing an antigen to which the antibody binds. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. Multivalent antibodies may comprise (or consist of) three to about eight, for example four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1).sub.n-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

### C. Antibody Conjugates

Antibodies of the present disclosure may be linked to at least one agent to form an antibody conjugate. The conjugate can be, for example, an antibody conjugated to another proteinaceous, carbohydrate, lipid, or mixed moiety molecule(s). Such antibody conjugates include, but are not limited to, modifications that include linking the antibody to one or more polymers. For example, an antibody may be linked to one or more water-soluble polymers. Linkage to a water-soluble polymer reduces the likelihood that the antibody will precipitate in an aqueous environment, such as a physiological environment. One skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, an enzyme (e.g., that catalyzes a colorimetric or fluorometric reaction), a substrate, a solid matrix, such as biotin. An antibody may comprise one, two, or more of any of these labels.

Antibody conjugates may be used to deliver cytotoxic agents to target cells. Cytotoxic agents of this type may improve antibody-mediated cytotoxicity, and include such moieties as cytokines that directly or indirectly stimulate cell death, radioisotopes, chemotherapeutic drugs (including prodrugs), bacterial toxins (e.g., Pseudomonas exotoxin, diphtheria toxin, etc.), plant toxins (e.g., ricin, gelonin, etc.), chemical conjugates (e.g., maytansinoid toxins, auristatins, α-amanitin, anthracyclines, calechaemicin, etc.), radioconjugates, enzyme conjugates (e.g., RNase conjugates, granzyme antibody-directed enzyme/prodrug therapy), and the like.

Antibody conjugates are also used as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

The paramagnetic ions contemplated for use as conjugates include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and bismuth (III).

The radioactive isotopes contemplated for use as conjugated include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred. Technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

The fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

Another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and may be used as antibody binding agents.

Derivatization of immunoglobulins by selectively introducing sulfhydryl groups in the Fc region of an immunoglobulin, using reaction conditions that do not alter the antibody combining site are also contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

### D. Antibody Drug Conjugates

Antibody drug conjugates, or ADCs, are a new class of highly potent biopharmaceutical drugs designed as a targeted therapy for the treatment of people with disease. ADCs are complex molecules composed of an antibody (a whole mAb or an antibody fragment, such as a scFv) linked, via a stable chemical linker with labile bonds, to a biological active cytotoxic/anti-viral payload or drug. Antibody drug conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the diseased cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on diseased cells). Antibodies track these proteins down in the body and attach themselves to the surface of the diseased cells. The biochemical reaction between the antibody and the target protein (antigen) triggers a signal in the targeted cell, which then absorbs or internalizes the antibody together with the cytotoxin. After the ADC is internalized, the cytotoxic drug is released and kills the cell or impairs cellular replication. Due to this targeting, ideally the drug has lower side effects and gives a wider therapeutic window than other agents.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and non-cleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (e.g., anti-cancer) drug. For example, a non-cleavable linker keeps the drug within the cell. As a result, the entire antibody, linker, and cytotoxic agent enter the targeted cell where the antibody is degraded to the level of amino acids. The resulting complex—amino acid, linker and cytotoxic agent—now becomes the active drug. In contrast, cleavable linkers are catalyzed by enzymes in the host cell, thereby releasing the cytotoxic agent.

Another type of cleavable linker adds an extra molecule between the cytotoxic drug and the cleavage site. This linker technology allows researchers to create ADCs with more flexibility without worrying about changing cleavage kinetics. Researchers are also developing a new method of peptide cleavage based on Edman degradation. Future direction in the development of ADCs also include the development of site-specific conjugation (TDCs) to further improve stability and therapeutic index and α-emitting immunoconjugates and antibody-conjugated nanoparticles.

### E. Production and Purification of Antibodies

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

Alternatively, a molecular cloning approach may be used to generate monoclonal antibodies. Single B cells labeled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

Monoclonal antibodies produced by any means may be purified, if desired, using filtration, centrifugation, and various chromatographic methods, such as FPLC or affinity chromatography. Fragments of the monoclonal antibodies of the disclosure can be obtained from the purified monoclonal antibodies by methods that include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

The antibodies of the present disclosure may be purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxyapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

### F. Modification of Antibodies

The sequences of antibodies may be modified for a variety of reasons, such as improved expression, improved cross-reactivity, or diminished off-target binding. Modified antibodies may be made by any technique known to those of skill in the art, including expression through standard molecular biological techniques, or the chemical synthesis of polypeptides.

For example, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

An amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

An isolated monoclonal antibody, or antigen binding fragment thereof, may contain a substantially homogeneous glycan without sialic acid, galactose, or fucose. The aforementioned substantially homogeneous glycan may be covalently attached to the heavy chain constant region.

A monoclonal antibody may have a novel Fc glycosylation pattern. Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

The isolated monoclonal antibody, or antigen binding fragment thereof, may be present in a substantially homogenous composition represented by the GNGN or G1/G2 glycoform, which exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. Fc glycosylation plays a significant role in anti-viral and anti-cancer properties of therapeutic mAbs. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, may be expressed in cells that express beta (1,4)-N-acetylglucosaminyltransferase III (GnT III), such that GnT III adds GlcNAc to the antibody. Methods for producing antibodies in such a fashion are provided in WO/9954342 and WO/03011878. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express monoclonal antibodies.

It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene comprising the cDNA encoding the antibodies.

Antibodies can be engineered to enhance solubility. For example, some hydrophilic residues such as aspartic acid, glutamic acid, and serine contribute significantly more favorably to protein solubility than other hydrophilic residues, such as asparagine, glutamine, threonine, lysine, and arginine.

B cell repertoire deep sequencing of human B cells from blood donors has been performed on a wide scale. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

Any suitable approach, including any of the above approaches, can be used to reduce or eliminate human immunogenicity of an antibody.

### G. Characterization of Antibodies

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody binds may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce monoclonal antibodies having the desired characteristics. MAP may be used to sort the antibodies of the disclosure into groups of antibodies binding different epitopes.

The present disclosure includes antibodies that may bind to the same epitope, or a portion of the same epitope. One can easily determine whether an antibody binds to the same epitope as, or competes for binding with, a reference antibody by using routine methods known in the art. For example, to determine if a test antibody binds to the same epitope as a reference antibody, the reference antibody is allowed to bind to the target molecule under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference antibody, the above-described binding methodology is performed in two orientations: In a first orientation, the reference antibody is allowed to bind to its antigen under saturating conditions followed by assessment of binding of the test antibody to the antigen. In a second orientation, the test antibody is allowed to bind to the antigen under saturating conditions followed by assessment of binding of the reference antibody to the antigen. If, in both orientations, only the first (saturating) antibody is capable of binding to the antigen, then it is concluded that the test antibody and the reference antibody compete for binding to the antigen. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90%, or even 99% as measured in a competitive binding assay (see, e.g., Junghans et al, Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In another aspect, the antibodies may be defined by their variable sequence, which include additional “framework” regions. These are provided in Tables 2, 3, 6, 9, and 10, that represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the antibodies provided herein and their antigen-binding fragments. A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art.

One can determine the biophysical properties of antibodies. One can use elevated temperature to unfold antibodies to determine relative stability, using average apparent melting temperatures. Differential Scanning Calorimetry (DSC) measures the heat capacity, Cp, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pls). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Generally, it is thought that autoreactive clones should be eliminated during ontogeny by negative selection; however, it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

### IV. PHARMACEUTICAL FORMULATIONS

The present disclosure provides pharmaceutical compositions comprising antibodies or antibody fragments with engineered Fc domains. Such compositions comprise a prophylactically or therapeutically effective amount of an antibody or a fragment thereof and a pharmaceutically acceptable carrier. In some aspects, the pharmaceutical composition will comprise one or more mRNAs encoding the antibody or antibody fragment. In the case of an antibody therapeutic, methods of delivering mRNA encoding the antibody to the patient has been described, for example, in U.S. Pat. No. 10,899,830, which is incorporated herein by reference in its entirety.

In the case that a nucleic acid molecule encoding a antibody with an engineered Fc domain is used in a pharmaceutical composition, the nucleic acid molecule may comprise or consist of deoxyribonucleotides and/or ribonucleotides, or analogs thereof, covalently linked together. A nucleic acid molecule as described herein generally contains phosphodiester bonds, although in some cases, nucleic acid analogs are included that may have at least one different linkage, e.g., phosphoramidate, phosphorothioate, phosphorodithioate, or O-methylphosphoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. A nucleic acid molecule may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A nucleic acid molecule is composed of a specific sequence of four nucleotide bases: adenine (A), cytosine (C), guanine (G), thymine (T), and uracil (U) for thymine when the polynucleotide is RNA. Thus, the term “nucleic acid sequence” is the alphabetical representation of a nucleic acid molecule. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

In some embodiments, the nucleic acids of the present disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties.

In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH3 (“OMe” or “O-methyl”), and 2′-O(CH2)2OCH3 (“MOE”). In certain embodiments, sugar substituents at the 2′ position is selected from allyl, amino, azido, thio, O-allyl, O-C1-C10 alkyl, O-C1-C10 substituted alkyl; OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl. Examples of sugar substituents at the 5-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties (see, e.g., PCT International Application WO 2008/101157, for additional 5′,2′-bis substituted sugar moieties and nucleosides).

Nucleosides comprising 2-substituted sugar moieties are referred to as 2′-substituted nucleosides. In some embodiments, a 2′-substituted nucleoside comprises a 2′-substituent group selected from halo, allyl, amino, azido, SH, CN, OCN, CF3, OCF3, O, S, or N(Rm)-alkyl; O, S, or N(Rm)-alkenyl; O, S or N(Rm)-alkynyl; O-alkylenyl-O-alkyl, alkynyl, alkaryl, aralkyl, O-alkaryl, O-aralkyl, O(CH2)2SCH3, O(CH2)2-O—N(Rm)(Rn) or O—CH2-C(═O)—N(Rm)(Rn), where each Rm and Rn is, independently, H, an amino protecting group or substituted or unsubstituted C1-C10 alkyl. These 2′-substituent groups can be further substituted with one or more substituent groups independently selected from hydroxyl, amino, alkoxy, carboxy, benzyl, phenyl, nitro (N02), thiol, thioalkoxy (S-alkyl), halogen, alkyl, aryl, alkenyl and alkynyl.

In some embodiments, a 2′-substituted nucleoside comprises a 2′-substituent group selected from F, NH2, N3, OCF3, O—CH3, O(CH2)3NH2, CH2-CH═CH2, O—CH2-CH═CH2, OCH2CH2OCH3, O(CH2)2SCH3, O—(CH2)2-O—N(Rm)(Rn), O(CH2)2O(CH2)2N(CH3)2, and N-substituted acetamide (O—CH2-C(═O)—N(Rm)(Rn) where each Rm and Rn is, independently, H, an amino protecting group or substituted or unsubstituted C1-C10 alkyl.

In some embodiments, a 2′-substituted nucleoside comprises a sugar moiety comprising a 2′-substituent group selected from F, OCF3, O—CH3, OCH2CH2OCH3, O(CH2)2SCH3, O(CH2)2-O—N(CH3)2, —O(CH2)2O(CH2)2N(CH3)2, and O—CH2-C(═O)—N(H)CH3.

In some embodiments, a 2′-substituted nucleoside comprises a sugar moiety comprising a 2′-substituent group selected from F, O—CH3, and OCH2CH2OCH3.

In some embodiments, nucleosides of the present disclosure comprise one or more unmodified nucleobases. In certain embodiments, nucleosides of the present disclosure comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, each of which is herein incorporated by reference in its entirety.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present disclosure involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. In some aspects, a nucleic acid molecule encoding an engineered antibody is a modified RNA, such as, for example, a modified mRNA. Modified (m)RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In some embodiments, the (m)RNA molecules used herein may have the uracils replaced with psuedouracils such as 1-methyl-3′-pseudouridylyl bases. In some embodiments, some of the uracils are replaced, but in other embodiments, all of the uracils have been replaced. The (m)RNA may comprise a 5′ cap, a 5′ UTR element, an optionally codon optimized open reading frame, a 3′ UTR element, and a poly(A) sequence and/or a polyadenylation signal.

The nucleic acid molecule, whether native or modified, may be delivered as a naked nucleic acid molecule or in a delivery vehicle, such as a lipid nanoparticle. A lipid nanoparticle may comprise one or more nucleic acids present in a weight ratio to the lipid nanoparticles from about 5:1 to about 1:100. In some embodiments, the weight ratio of nucleic acid to lipid nanoparticles is from about 5:1, 2.5:1, 1:1, 1:5, 1:10, 1:15, 1:20, 1:25, 1:30, 1:35, 1:40, 1:45, 1:50, 1:60, 1:70, 1:80, 1:90, or 1:100, or any value derivable therein.

In some embodiments, the lipid nanoparticles used herein may contain one, two, three, four, five, six, seven, eight, nine, or ten lipids. These lipids may include triglycerides, phospholipids, steroids or sterols, a PEGylated lipids, or a group with a ionizable group such as an alkyl amine and one or more hydrophobic groups such as C6 or greater alkyl groups.

In some aspects of the present disclosure, the lipid nanoparticles are mixed with one or more steroid or a steroid derivative. In some embodiments, the steroid or steroid derivative comprises any steroid or steroid derivative. As used herein, in some embodiments, the term “steroid” is a class of compounds with a four ring 17 carbon cyclic structure, which can further comprises one or more substitutions including alkyl groups, alkoxy groups, hydroxy groups, oxo groups, acyl groups, or a double bond between two or more carbon atoms.

In some aspects of the present disclosure, the lipid nanoparticles are mixed with one or more PEGylated lipids (or PEG lipid). n some embodiments, the present disclosure comprises using any lipid to which a PEG group has been attached. In some embodiments, the PEG lipid is a diglyceride which also comprises a PEG chain attached to the glycerol group. In other embodiments, the PEG lipid is a compound which contains one or more C6-C24 long chain alkyl or alkenyl group or a C6-C24 fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present disclosure are taught by U.S. Pat. No. 5,820,873, WO 2010/141069, or U.S. Pat. No. 8,450,298, which is incorporated herein by reference.

In some aspects of the present disclosure, the lipid nanoparticles are mixed with one or more phospholipids. In some embodiments, any lipid which also comprises a phosphate group. In some embodiments, the phospholipid is a structure which contains one or two long chain C6-C24 alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some aspects of the present disclosure, lipid nanoparticle containing compounds containing lipophilic and cationic components, wherein the cationic component is ionizable, are provided. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C6-C24 alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some aspects of the present disclosure, composition containing compounds containing lipophilic and cationic components, wherein the cationic component is ionizable, are provided. In some embodiments, ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C6-C24 alkyl or alkenyl groups, but may have at least 1 or more that 6 tails.

In some embodiments, the amount of the lipid nanoparticle with the nucleic acid molecule encapsulated in the pharmaceutical composition is from about 0.1% w/w to about 50% w/w, from about 0.25% w/w to about 25% w/w, from about 0.5% w/w to about 20% w/w, from about 1% w/w to about 15% w/w, from about 2% w/w to about 10% w/w, from about 2% w/w to about 5% w/w, or from about 6% w/w to about 10% w/w. In some embodiments, the amount of the lipid nanoparticle with the nucleic acid molecule encapsulated in the pharmaceutical composition is from about 0.1% w/w, 0.25% w/w, 0.5% w/w, 1% w/w, 2.5% w/w, 5% w/w, 7.5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, to about 95% w/w, or any range derivable therein.

In some aspects, the present disclosure comprises one or more sugars formulated into pharmaceutical compositions. In some embodiments, the sugars used herein are saccharides. These saccharides may be used to act as a lyoprotectant that protects the pharmaceutical composition from destabilization during the drying process. These water-soluble excipients include carbohydrates or saccharides such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol.

In some embodiments, the amount of the sugar in the pharmaceutical composition is from about 25% w/w to about 98% w/w, 40% w/w to about 95% w/w, 50% w/w to about 90% w/w, 50% w/w to about 70% w/w, or from about 80% w/w to about 90% w/w. In some embodiments, the amount of the sugar in the pharmaceutical composition is from about 10% w/w, 15% w/w, 20% w/w, 25% w/w, 30% w/w, 35% w/w, 40% w/w, 45% w/w, 50% w/w, 52.5% w/w, 55% w/w, 57.5% w/w, 60% w/w, 62.5% w/w, 65% w/w, 67.5% w/w, 70% w/w, 75% w/w, 80% w/w, 82.5% w/w, 85% w/w, 87.5% w/w, 90% w/w, to about 95% w/w, or any range derivable therein.

In some embodiments, the pharmaceutically acceptable polymer is a copolymer. The pharmaceutically acceptable polymer may further comprise one, two, three, four, five, or six subunits of discrete different types of polymer subunits. These polymer subunits may include polyoxypropylene, polyoxyethylene, or a similar subunit. In particular, the pharmaceutically acceptable polymer may comprise at least one hydrophobic subunit and at least one hydrophilic subunit. In particular, the copolymer may have hydrophilic subunits on each side of a hydrophobic unit. The copolymer may have a hydrophilic subunit that is polyoxyethylene and a hydrophobic subunit that is polyoxypropylene.

In some aspects, the present disclosure provides pharmaceutical compositions that contain one or more salts. The salts may be an inorganic potassium or sodium salt such as potassium chloride, sodium chloride, potassium phosphate dibasic, potassium phosphate monobasic, sodium phosphate dibasic, or sodium phosphate monobasic. The pharmaceutical composition may comprise one or more phosphate salts such to generate a phosphate buffer solution. The phosphate buffer solution may be comprise each of the phosphates to buffer a solution to a pH from about 6.8, 6.9, 7.0, 7.1, 7.2, 7.3, 7.4, 7.5, 7.6, 7.7, 7.8, 7.9, or 8.0, or any range derivable therein.

In some aspects, the present disclosure comprises one or more excipients formulated into pharmaceutical compositions. An “excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Furthermore, these compounds may be used as diluents in order to obtain a dosage that can be readily measured or administered to a patient. Non-limiting examples of excipients include polymers, stabilizing agents, surfactants, surface modifiers, solubility enhancers, buffers, encapsulating agents, antioxidants, preservatives, nonionic wetting or clarifying agents, viscosity increasing agents, and absorption-enhancing agents.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and can preferably include an adjuvant. Water is a particular carrier when the pharmaceutical composition is administered by injections, such an intramuscular injection. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Therapeutic proteins and mRNAs of the present disclosure, as described herein, can be formulated for parenteral administration, e.g., formulated for injection via the intradermal, intravenous, intramuscular, subcutaneous, intra-tumoral or even intraperitoneal routes. The antibodies could alternatively be administered by a topical route directly to the mucosa, for example by nasal drops, inhalation, or by nebulizer. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Dosage can be by a single dose schedule or a multiple dose schedule. Multiple doses may be used in a primary immunization schedule and/or in a booster immunization schedule. In a multiple dose schedule the various doses may be given by the same or different routes. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.).

The compositions disclosed herein may be used to treat both children and adults. Thus, a human subject may be less than 1 year old, 1-5 years old, 5-16 years old, 16-55 years old, 55-65 years old, or at least 65 years old.

Preferred routes of administration include, but are not limited to, intramuscular, intraperitoneal, intradermal, subcutaneous, intravenous, intraarterial, and intraoccular injection. Particularly preferred routes of administration include intramuscular, intradermal and subcutaneous injection.

### V. METHODS OF TREATMENT

Certain aspects of the present embodiments can be used to prevent or treat a disease or disorder, such as cancer, such as lung cancer, prostate cancer, stomach cancer, thyroid cancer, or breast cancer.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody or antibody fragment with an engineered Fc domain, either alone or in combination with administration of chemotherapy, immunotherapy, or radiotherapy, performance of surgery, or any combination thereof.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, a neurodegenerative disease, and/or a genetic disorder).

In certain embodiments, the compositions and methods of the present embodiments involve an antibody or an antibody fragment with an engineered Fc domain, in combination with a second or additional therapy, such as chemotherapy or immunotherapy. For example, the disease may be a cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials & Methods

Cloning for Fc display on CHO-T cells. The hinge, CH2, and CH3 regions of the human IgG1 heavy chain were PCR amplified by Q5 Polymerase (New England Biolabs #M0491S) from the AbVec-hu4D5 plasmid (Nguyen et al., 2018) using primers #1 and #3. The PDGFR sequence (Gronwald et al., 1988) was amplified from pPyFab display (Nguyen et al., 2018) with primers #4 and #5. The two PCR products were annealed, amplified with primers #5 and #2, then introduced into the pPyEBV plasmid (Kunaparaju et al., 2005) using the KpnI and BamHI restriction sites to create the pPy-FcDisp plasmid. Primers are listed in Table 3.

Flow cytometry scanning of CHO-T cells displaying Fc. CHO-T cells were grown in CHO-S-SFM-II media supplemented with 2× Glutamax (Gibco #35050061). 4.5×10⁶ cells were transfected either with 12.5 μg of blank pPyEBV or pPy-FcDisp plasmids using Lipofectamine 2000 (Thermo Fisher Scientific #11668500) following the manufacturer's instruction. Cells were spun down and resuspended into new media one day after transfection. Two days after transfection, cells were washed with 1 mL flow buffer (OptiMEM+0.5% BSA) and incubated for 30 min at 4° C. with either 1:1000 goat-anti-human Fc-Alexa Fluor 647 or 50 nM FcγRIIIa-SA PE.

The monomeric FcγRIIIa reagent for cell staining was generated by incubating biotinylated FcγRIIIa (V158; Sino Biological #10389-H27H1-B) with fluorescent streptavidin overnight at 4° C. To generate a monomeric reagent, a molar ratio of 1:7:2 FcγRIIIa:biotin:streptavidin was used, so that <10% of the final product is expected to have >1 FcγRIIIa per streptavidin, based on a Poisson distribution. Samples were washed, resuspended in flow buffer, and scanned by flow cytometry using a BD Fortessa. Data were analyzed in Flowjo v10.7.1, live cell gates were drawn based on FSC vs. SSC profiles, and only this population was used for determination of mean fluorescence intensity (MFI) values.

Creation of the Fc library. Diversity was introduced into the Fc at ten positions as indicated in Table 1 using degenerate overlapping 40-mer DNA oligomers (Sigma-Aldrich; Table 3) spanning from the KpnI restriction site before the signal sequence to an XhoI restriction site within the Fc gene. The XhoI restriction site (CTCGAG) which was introduced into the genes coding for Fc residues P343/R345/E346 (CCTCGAGAA). Assembly PCR with 4 μM of each 40-mer and Q5 Polymerase (New England Biolabs) was used to create the mutated Fc fragments, the assembled DNA was amplified with primers FCLibF01 and FCLibR12 by Q5 Polymerase, gel purified and digested, then ligated into pPy-FcDisp using the KpnI and XhoI restriction sites.

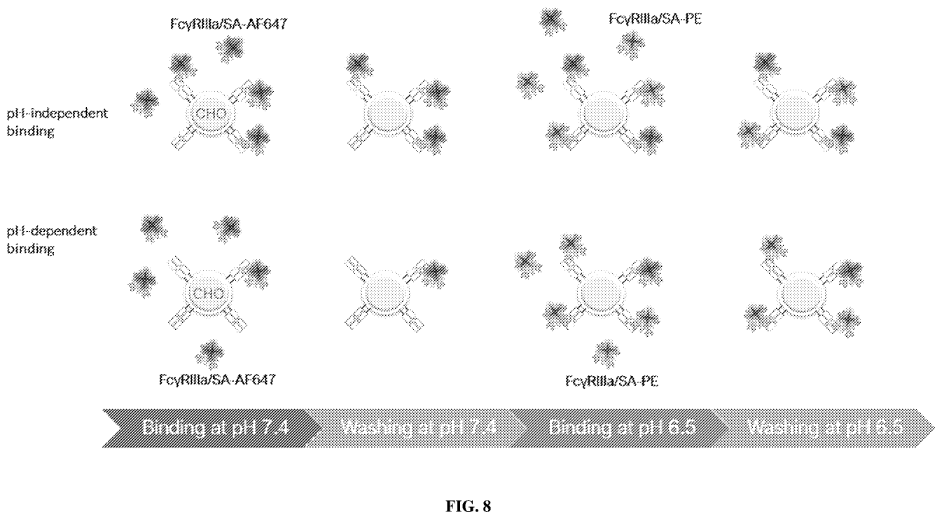

Screening of the Fc library. Library plasmid DNA, blank pPyEBV, pPyFcDisp, pPyFcDisp-LALAPG were transfected to CHO-T cells doped with a blank carrier plasmid as previously described (Nguyen et al., 2018). Cells were then grown at 37° C. overnight, spun at 200×g for 5 min and resuspended in fresh media (CHO-S-SFM-II media supplemented with 2× Glutamax). Two days after transfection, cells were collected again and resuspended in fresh media containing 150 μg/ml Hygromycin B to maintain the episome. Five days after transfection, fresh media containing 300 g/ml Hygromycin B was provided and maintained for subsequent steps. The cells were cultured for two weeks of growth to allow killing of cells lacking pPy episomes, while maintaining total cell numbers >5 times the library size. Two weeks after transfection, cells were collected and subjected to FACS. For the first round, 7×10⁶ cells were screened for binding to 50 nM FcγRIIIa-SA-PE. After sorted cells has grown up, 1×10⁷ cells were screened for binding to 20 nM FcγRIIIa-SA-PE. For the last two rounds, 5×10⁶ cells were subjected to a dual staining process prior to FACS (FIG. 8). Cells were first stained with 50 nM monomeric FcγRIIIa-SA-AF647 in flow buffer (OptiMEM+0.5% BSA) at pH 7.4. After washing, the cells were then incubated with 20 nM monomeric FcγRIIIa-SA-PE in flow buffer (OptiMEM+0.5% BSA) adjusted to pH 6.5. In the first two rounds of FACS, all cells with PE signal higher than that observed for the negative control LALAPG Fc were sorted into warm media and grown for 7 days. For the following two rounds of sorting, cells with PE signal greater than that observed for WT at the same AF647 signal were collected.

Isolation, expression, and purification of Fc variants with hu4D5 Fab arms. After each round of cell sorting and growth, ˜10⁶ cells were collected for DNA purification using a Genomic DNA Purification Kit (Invitrogen #K182002). This was then used as template to amplify the randomized Fc region using primers FCLibF01 and FCLibR12 and ligated into the pPyFcDisp backbone via KpnI and XhoI restriction sites. After transformation into E. coli, 39 individual colonies were isolated for sequencing. To express Fc variants as full-length antibodies, the entire hinge, CH2, and CH3 region were amplified from pPyFcDisp, and inserted into AbVec vector (Nguyen et al., 2018) encoding the hu4D5 heavy chain using Gibson assembly. After sequence confirmation, the plasmid was midi-prepped (Zymo Research #D4200) and co-transfected with plasmid encoding the hu4D5 light chain at a 1:1 ratio into 25 mL of ExpiCHO cells following the manufacturer's instruction. After 7 days of expression, media were harvested and antibody purified by protein A followed by preparative size exclusion chromatography on a Superdex S200 column on an Akta FPLC.

Characterization of hu4D5-Fc variant binding to human Fcγ receptors. To express human Fcγ receptors, pcDNA3.1 plasmids containing the genes with N-term AVI and C-term His tags were transfected into ExpiHEK cells using manufacturer instructions and purified by immobilized metal affinity chromatography (Qiagen #30210). The FcγRIIIa V158 and F158 receptors were biotinylated using BirA (Avidity) and further purified by Superdex S200 size exclusion chromatography column with an Akta FPLC. Receptors FcγRIIa R131, FcγRIIa H131 and FcγRIIb were purified by SEC without biotinylation. Plasmids and GST-tagged FcRn proteins (Berntzen et al., 2005) were provided by George Georgiou, University of Texas at Austin.

For ELISA, 96-well high-binding plates were coated with 2 g/mL antibody in PBS at 4° C. overnight. Wells were then blocked using 5% BSA in PBS with 0.05% Tween-20 (PBS-T) at room temperature for an hour, washed, then incubated with duplicate serial dilutions of FcγRIIIa in PBS-T adjusted to pH 6.5 or 7.4 for an hour. Wells were washed three times using PBS-T at the specified pH and captured FcγRIIIa detected with 1:1000 anti-His-HRP (Genscript Biotech #A00612). After another one-hour incubation and triplicate PBS-T wash, 50 μL TMB substrate (Thermo Scientific) was added per well followed by 50 μL of 1N HCl to quench the reaction and the absorbance at 450 nm recorded on a SpectraMax M5. For FcRn ELISA, anti-FLAG-HRP (Sigma-Aldrich #A-8592) was used for detection. Data were fit to four-parameter curves with Graphpad.

For affinity measurements via BLI using SA biosensors, tips were prewetted in phosphate-buffered saline (PBS) for 10 min, then dipped into wells containing 1 μg/mL monomeric biotinylated FcγRIIIa in PBS until a shift of >0.25 nm was achieved. The sensors were then dipped into wells containing kinetic buffer (PBS+0.02% Tween20+0.1% BSA) adjusted to pH 7.4 or 6.5 for 180 sec. Antibody association signals were recorded by dipping sensors into wells containing kinetic buffer and hu4D5-Fc variants in concentrations ranging from 62.5 nM to 2 M for 60 sec. Dissociation signals were recorded by dipping sensors into wells with kinetic buffer for 120 sec. For affinity measurements via BLI using FAB2G biosensors, antibody variants were captured on FAB2G tips until shift of 3 nm was reached, association (30 sec) and dissociation (30 sec) rates were measured with serially diluted FcγRs. Association and dissociation constants were fitted from 1:1 association then dissociation model in GraphPad using the full association step and the initial 5 seconds of dissociation. Equilibrium Ko values were calculated from a Langmuir isotherm: R_{eq}=Rₘₐₓ*C/(K_{d}+C) where R_{eq} is the equilibrium response at each antibody concentration C, and Rₘₐₓ is the maximum specific binding response obtained from fitting. Statistical significance was determined by t test in GraphPad.

ADCC assay. Target SKBR3 (ATCC #HTB-30) and SKOV3 (ATCC #HTB-77) cells were cultured in DMEM medium supplemented with 10% FBS. Effector NK-92 cells stably expressing FcγRIIIa allele V158 (ATCC #PTA 6967) cells were cultured in Alpha Minimum Essential medium without ribonucleosides and deoxyribonucleosides (Sigma-Aldrich #M0200) but with 2 mM L-glutamine and 1.5 g/L sodium bicarbonate, supplemented with 0.2 mM inositol, 0.1 mM 2-mercaptoethanol, 0.02 mM folic acid, 200 U/ml recombinant IL-2, 12.5% horse serum, and 12.5% fetal bovine serum. For the ADCC assay, target cells were collected by centrifugation at 300×g for 5 min, washed in PBS and labeled with 2 μM Calcein-AM (BD Pharmingen #564061) in DMEM at 37° C. for 30 min. Calcein-loaded target cells were washed twice and resuspended in culture media (DMEM with 10% FBS pH adjusted to pH 6.5 or 7.4 by addition of hydrochloric acid and 20 mM MOPS), and seeded at 10,000 cells/well in 100 uL in a 96-well plate. Antibody hu4D5-Fc variants were serially diluted in 20 mM MOPS buffered saline at pH 6.5 or pH 7.4 and 50 μL added per well. NK92 effector cells resispended in the same culture media were added to the wells at 100,000 cells/well in 50 μL for a final E:T ratio of 10:1 and incubated at 37° C. for 4 hr. Plates were then centrifuged again to remove cells from the media. Calcein released in the media was detected by fluorescence at excitation and emission wavelengths of 485 and 525 nm, respectively. The percent of target cells lysed was calculated as follows: 100%×(E-S)/(M-S), where E is the fluorescence of experimental well, S is the fluorescence in the absence of antibody resulting from non-specific lysis, and M is the maximum fluorescence after treatment of target cells with lysis buffer (Triton X-100 at 2% v/v, SDS 1% w/v, 100 mM NaCl, and 1 mM EDTA). For each experiment, data were normalized to the mean percent lysis for the highest antibody concentration. Curves were then fit to four parameter logistic curves in GraphPad to determine EC₅₀ values and 90% confidence intervals. Selectivity was calculated as the ratio of the EC₅₀ at pH 7.4 over the EC₅₀ at pH 6.5, with statistical significance determined by two-sided t-test in GraphPad.

Murine pharmacokinetic studies. All animal procedures were performed in a facility accredited by the Association for Assessment and Accreditation of Laboratory Animal Care International in accordance with protocols approved by UT Austin (#2019-00226) Animal Care and Use Committees and the principles outlined in the Guide for the Care and Use of Laboratory Animals.

Pharmacokinetic studies were performed in homozygous transgenic Tg32 mice expressing human FcRn under the human promoter (The Jackson Laboratory Cat #014565). Mice were administered 2 mg/kg of 4D5 antibody Fc variants at 5-6 weeks of age by intraperitoneal injection. Blood from the lateral tail vein was collected every 3-4 days and used in ELISA to determine the serum antibody concentration. High-binding 96-well plates (Corning) were coated overnight with 0.5 g/mL chimeric Her2-Fc (R&D Systems), then blocked with 5% milk in PBS-T and incubated with diluted serum samples (1:1000-1:100 depending on the time point) or purified hu4D5 antibody diluted with 1:100 mouse serum in duplicate. Human antibodies were detected with goat anti-human kappa light chain antibody-HRP (Southern Biotech, 1:2000 dilution). Absorbance at 450 nm was measured after application of TMB substrate (Thermo Scientific) and neutralization with 1 M HCl. A four-parameter fit for each standard curve was generated in GraphPad and used to quantify the anti-Her2 human antibody present. The beta-phase elimination constant (kₑ) was determined by log-linear regression of the concentration data, including at least six time points with measurable concentrations. Beta-phase half-life was determined from t_{β1/2}=ln 2/kₑ. Power analysis of the observed half-lives was performed with G*Power using alpha level of 0.05 and desired power of 0.9.

TABLE 3
List of primers used in this study. Degenerate codons used for library
primers: W (A or T), S (C or G), M (A or C), K (G or T), R (A or G), Y
(C or T), B (C or G or T), D (A or G or T), H (A or C or T), V (A or C or
G), N (any base).

| Primer | | SEQ |
|---|---|---|
| Name | 5′ to 3′ sequence | ID NO: |
| #1 | ATACTATTCCTGGTAGCAACCGCTACAGGAGTACATTCCGAGCC | 11 |
| | CAAATCTTGTGACAAAAC | |
| #2 | GCTCGTGGTACCGCGCGCCTAAATGGGTTGAACCATGGGCTGGT | 12 |
| | CCTGCATCATACTATTCCTGGTAGCAACCGCTAC | |
| #3 | ACCGGAACCGCCTCCTGAACCTCCACCTTTACCCGGGGACAGG | 13 |
| #4 | GGAGGCGGTTCCGGTAATGCTGTGGGCCAGG | 14 |
| #5 | GCTCGTGGATCCCTAACGTGGCTTCTTCTG | 15 |
| FCLibF01 | GCTCGTGGTACCGCGCGCCTAAATGGGTTGAACCATGGGC | 16 |
| FCLibF02 | TGGTCCTGCATCATACTATTCCTGGTAGCAACCGCTACAG | 17 |
| FCLibF03 | GAGTACATTCCGAGCCCAAATCTTGTGACAAAACTCACAC | 18 |
| FCLibF04 | ATGCCCACCGTGCCCAGCACCISAWSWSSWKGRSGRWCCG | 19 |
| FCLibF05 | TCAGTCTTCCTCTTCCCCCCAAAACCCAAGGACACCCTCA | 20 |
| FCLibF06 | TGATCTCCCGGACCCCTGAGGTCACATGCGTGGTGGTGGA | 21 |
| FCLibF07 | CGTGRRSBMCGAAGACCCTGAGGTCAAGTTCAACTGGTAC | 22 |
| FCLibF08 | GTGGACGGCGTGGAGGTGCATAATGCCAAGACAAAGCCGC | 23 |
| FCLibF09 | GGGAGGAGCAGYACAACMRCACGTACCGTGTGGTCAGCGT | 24 |
| FCLibF10 | CCTCACCGTCCTGCACCAGGACTGGCTGAATGGCAAGGAG | 25 |
| FCLibF11 | TACAAGTGCAAGGTCTCCAACAAAGMSCTCCCAGCCCCCA | 26 |
| FCLibF12 | TCGAGAAAACCATCTCCAAAGCCAAAGGGCAGCCTCGAGA | 27 |
| FCLibR01 | AATAGTATGATGCAGGACCAGCCCATGGTTCAACCCATTT | 28 |
| FCLibR02 | TTTGGGCTCGGAATGTACTCCTGTAGCGGTTGCTACCAGG | 29 |
| FCLibR03 | GTGCTGGGCACGGTGGGCATGTGTGAGTTTTGTCACAAGA | 30 |
| FCLibR04 | GGGGGGAAGAGGAAGACTGACGGWYCSYCMWSSWSWTSAG | 31 |
| FCLibR05 | CTCAGGGGTCCGGGAGATCATGAGGGTGTCCTTGGGTTTT | 32 |
| FCLibR06 | CAGGGTCTTCGKVSYYCACGTCCACCACCACGCATGTGAC | 33 |
| FCLibR07 | TGCACCTCCACGCCGTCCACGTACCAGTTGAACTTGACCT | 34 |
| FCLibR08 | GYKGTTGTRCTGCTCCTCCCGCGGCTTTGTCTTGGCATTA | 35 |
| FCLibR09 | CCTGGTGCAGGACGGTGAGGACGCTGACCACACGGTACGT | 36 |
| FCLibR10 | TTGGAGACCTTGCACTTGTACTCCTTGCCATTCAGCCAGT | 37 |
| FCLibR11 | TTTGGAGATGGTTTTCTCGATGGGGGCTGGGAGSKCTTTG | 38 |
| FCLibR12 | GCAGGGTGTACACCTGTGGTTCTCGAGGCTGCCCTTTGGC | 39 |

### Example 2—CHO Cell Display Discriminates Among Fc Variants with Different Fc; RIIIa Affinities

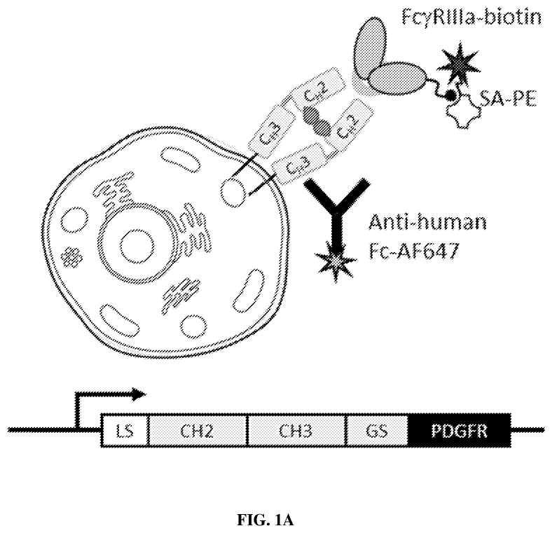

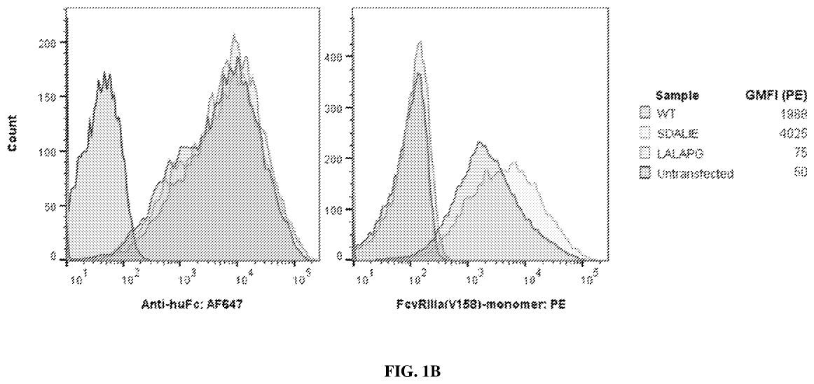

For Fc engineering, a mammalian cell display system, which allows for engineering on the same cell line used for manufacturing, was selected (Nguyen et al., 2018; Wagner et al., 2019). Chinese hamster ovary (CHO) cells preserve the essential glycosylation at position N297 that supports binding to classical Fcγ receptors and can modulate Fc effector functions independently of residue changes (Nimmerjahn & Ravetch, 2012). To first determine the display level and functionality of Fc expressed on the CHO cell surface, residues 216-447 (EU numbering) of the human IgG1 Fc domain, corresponding to the complete hinge, CH2 and CH3 domains, with an N-terminal murine IgK leader sequence, were cloned into the pPyEBV vector previously used for Fab and TCR display on CHO cells (Nguyen et al., 2018; Wagner et al., 2019) (FIG. 1A). The expressed homodimeric Fc was anchored to the CHO cell surface by a (Gly₃Ser)₂ linker and PDGFR transmembrane region at the C-terminal end of the CH3 domain. A modified Kozak sequence was used to reduce Fc expression level (Ferreira et al., 2013) and thereby modulate avidity effects (Nguyen et al., 2018).

The wild-type human IgG1 Fc and variants with greatly reduced (LALAPG) (Lo et al., 2017) or improved (SDALIE) (Lazar et al., 2006) FcγRIIIa binding were cloned into the display construct. After sequence confirmation, midi-prepped plasmid DNA was transiently transfected into CHO-T cells for semi-stable plasmid maintenance. After hygromycin-B selection, Fc display levels were monitored by anti-human Fc-Alexa Fluor 647 (AF647) and biotinylated FcγRIIIa allele V158 monomerically bound to streptavidin-PE (FIG. 1A). Staining with anti-human Fc and FcγRIIIa was performed separately to avoid interference between the receptor and the anti-Fc antibodies. Flow cytometry showed similar high display levels for all three Fc variants displayed on the surface of CHO cells (FIG. 1B, left plot). Consistent with reported affinities (Lo et al., 2017; Lazar et al., 2006), SDALIE showed higher FcγRIIIa staining than wild-type, while LALAPG showed no FcγRIIIa staining at all (FIG. 1B, right plot). These results indicate that the CHO display system displays functional Fc variants and is able to distinguish among Fes with known FcγRIIIa affinity differences. Accordingly, this system was found to be suitable for selection of Fc variants with different FcγRIIIa binding characteristics.

### Example 3—Creation of an Fc Library Targeting the CH1-CH2 Hinge Region

Previous efforts to engineer pH-sensitive protein-protein interactions guided this work. The naturally pH-dependent interaction between human IgG1 Fc and the neonatal Fc receptor (FcRn) has been engineered to adjust antibody in vivo half-lives (Challa et al., 2014; Lee et al., 2019; Kroetsch et al., 2019), while novel pH-dependent binding has been introduced into other binding partners via histidine scanning mutagenesis (Sulea et al., 2020; Sarkar et al., 2002). In both cases, pH sensitivity relies on the presence of ionizable histidines with a pKₐ of ˜6.0 in the binding interface, which can be modulated by adjacent residues. When histidines within the paratope and/or epitope are protonated by an acidic environment, they can mediate interactions with negatively charged or polar residues on a binding partner; these interactions are lost at neutral pH when histidines are not protonated.

To guide library design, the Fc-FcγRIIIa crystal structure (Sondermann et al., 2000; Mizushima et al., 2011) was first examined, which revealed an asymmetric FcγRIIIa footprint on the Fc homodimer near the CH1-CH2 hinge region (FIG. 2A). The Fc-FcγRIIIa interactions are dominated by van der Waals contacts, including P329 on one chain (here called chain B), which forms a “proline sandwich” with W87 and W110 of the receptor (Sondermann et al., 2000). However, ˜6 potential hydrogen bonds are also present (Sondermann et al., 2000), primarily involving the other Fc chain (here called chain A), which may be amenable to engineering for pH-selective binding. Notably, if FcγRIIIa approaches the opposite Fc face, these interactions are reversed with chain B dominating the charge interactions and chain A participating in the “proline sandwich.” The chain A-receptor interface includes two FcγRIIIa histidine residues (H134 and H135) and one Fc histidine (H268; FIG. 2B). Residues H134 and H135 are in close proximity to multiple Fc residues, with H134 able to hydrogen bond with D265. Fc residue H268 is near FcγRIIIa K131 but no electrostatic interactions form between these residues.

To support formation of new direct charge-charge interactions, six Fc residues within 6.5 Å of the FcγRIIIa histidines (L234, L235, G236, G237, S267 and A327) were selected and allowed to remain unchanged or be substituted with negatively charged glutamic or aspartic acid residues with pKₐ values near 4 that likely retain negative charges at tumor-typical pH values. To introduce new histidine residues, three Fc residues within 5 Å of polar FcγRIIIa residues (E233, Y296 and S298) were identified for histidine scanning, while the existing H268 was allowed to remain a histidine or be substituted with Y/A/D/S to cover the chemical diversity compatible with protein-protein interactions with few codons (Fellouse et al., 2004).

This diversity was introduced into the Fc gene using primers with degenerate codons and overlap-extension PCR. At some sites, the degenerate codons introduced additional diversity beyond the intended changes (Table 1) resulting in a theoretical library size of 6×10⁶ (DNA) and 1.1×10⁶ (protein) variants. Amplified Fc genes were digested with KpnI and XhoI, ligated into similarly digested pPyEBV vector containing Fc with a premature stop codon to prevent expression from background plasmid, and transformed into E. coli to achieve an actual library size of ˜1×10⁷ transfectants. Sequencing of 10 colonies revealed 10 unique DNA sequences with three containing frameshifts, which is typical for PCR-generated libraries, and no unmodified background sequences, indicating that the actual library size is similar to the theoretical DNA library size. The designed primers allowed for simultaneous mutations, and the seven intact sequences each contained more than five different mutations.

TABLE 1
Amino acid sequences allowed at each targeted residues and variant
mutations. Wildtype residues are represented as regular letters,
and mutations introduced are represented by bold letters.

| Residue # | 233 | 234 | 235 | 236 | 237 | 267 | 268 | 296 | 298 | 327 |
|---|---|---|---|---|---|---|---|---|---|---|
| Wildtype | E | L | L | G | G | S | H | Y | S | A |
| Library | H | E | E | E | E | E | D | H | H | E |
| | Q | D | D | D | D | D | S | Y | N | D |
| | D | Q | Q | G | G | K | Y | | R | A |
| | E | H | H | | | G | A | | S | |
| | | V | V | | | N | P | | | |
| | | L | L | | | R | H | | | |
| | | | | | | S | | | | |
| 3A | D | V | V | G | G | E | D | H | S | A |
| 3E | D | V | V | G | G | D | D | Y | R | A |
| 3F | D | V | D | G | G | G | D | H | S | A |
| 4A | D | V | D | G | G | E | D | H | S | A |
| Acid-Fc | E | L | L | G | G | E | D | H | S | A |
| 3E2 | E | L | L | G | G | D | D | Y | R | A |
| 3F2 | E | L | L | G | G | G | D | H | S | A |

### Example 4—Selection of Fc Variants with pH-Dependent Binding

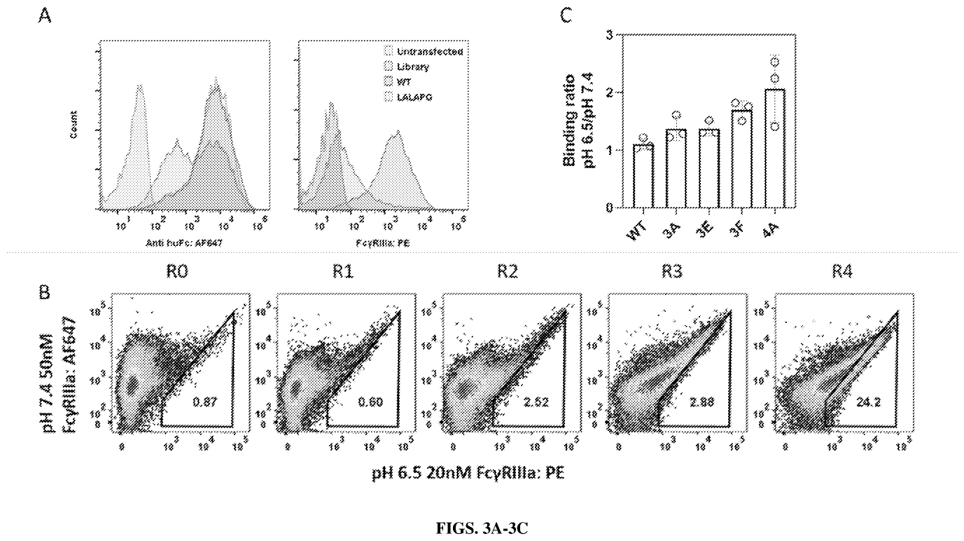

The library was transfected into CHO-T cells with carrier plasmid as previously described (Nguyen et al., 2018) to ensure each cell expressed at most one Fc variant. To further ensure that every library member is represented, we transfected 4.5×10⁷ CHO-T cells. Assuming a 30% transfection efficiency, which we typically observed for this system, ˜2 copies of each E. coli transfectant was present in the final CHO cell library. After hygromycin B selection, cells were stained with anti-human Fc-AF647 and monomeric PE-labeled FcγRIIIa (V158) separately at neutral pH and scanned by flow cytometry. Many changes introduced into the Fc region are likely detrimental to Fc folding, expression or FcγRIIIa binding. Consistent with this expectation, the library exhibited bi-phasic high and low display levels. Most library variants lost binding to FcγRIIIa, although a long tail overlapping with the wild-type FcγRIIIa binding profile suggested some members retain strong FcγRIIIa binding (FIG. 3A).

The library (7×10⁶ clones) was sorted by fluorescence-activated cell sorting (FACS) for two rounds to first isolate clones retaining binding to FcγRIIIa allele V158 at pH 6.5. The library was then subjected to a dual-color staining process for two additional sorting rounds to enrich for clones with stronger FcγRIIIa binding at pH 6.5 than at pH 7.4 (FIG. 8). In this process, cells were first labeled with 50 nM of AF647-labeled monomeric FcγRIIIa at pH 7.4, and then washed with flow buffer at pH 7.4 to allow clones binding weakly at neutral pH to dissociate. The cells were then stained with PE-labeled monomeric FcγRIIIa at pH 6.5, washed with flow buffer at pH 6.5 and sorted by FACS to collect clones strongly binding at low pH (high PE and low AF647 fluorescence). Comparison of populations from each round showed enrichment for improved FcγRIIIa binding as well as pH-dependence (FIG. 3B).

After each round of FACS, genomic DNA was extracted from the sorted cells. The pooled Fc sequences were PCR amplified, re-cloned en masse into the Fc display plasmid, transformed into E. coli, and plasmids from single colonies sequenced. Analysis of 23 colonies isolated from the third sorting round (R3) and 16 colonies from the fourth sorting round (R4) revealed several unique sequences. Four variants (3A, 3E, 3F, and 4A) were selected for further investigation based on the frequency of their appearance in R3 and R4 (Table 1), with 3A dominating rounds R3 (47.8%) and R4 (50%). All four variants contained six residue changes, with convergent E233D, L234V, H268D substitutions and conservation of the wild-type glycine residues at positions 236 and 237. In a prior structural study, G236 and G237 were shown to have strict psi/phi angles that cannot be achieved by other amino acids (Sondermann et al., 2000). These residues were previously shown to be crucial for FcγR binding (Brinkhaus et al., 2021), suggesting that selection preserves known structural constraints.

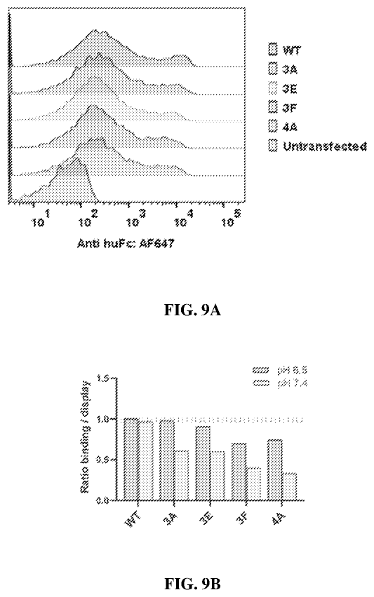

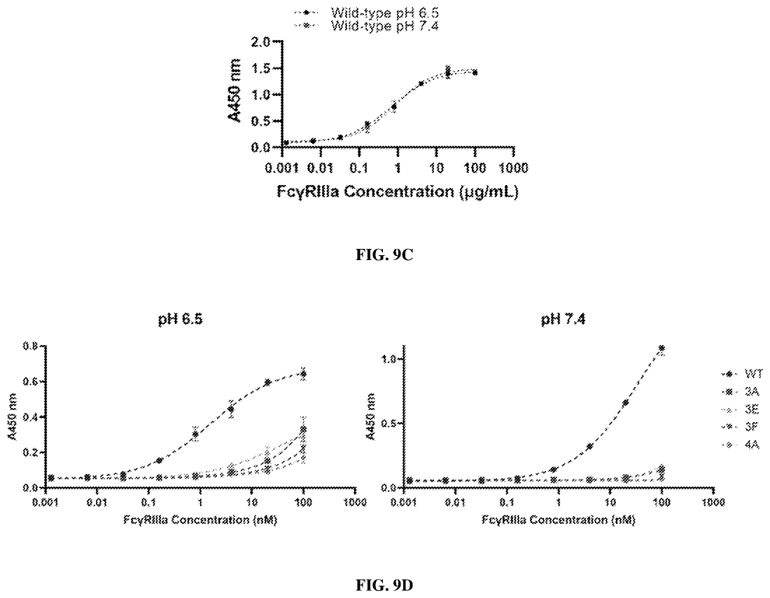

After transfection into CHO-T cells for analysis as monoclonal cell populations, all four variants exhibited similar display levels as wild-type Fc (FIG. 9A). While wild-type Fc showed similar binding levels to FcγRIIIa at pH 6.5 and pH 7.4, binding for all variants at pH 7.4 was reduced compared to binding at pH 6.5 (FIG. 9B). Variant pH-selectivity was determined as the ratio of the percent of cells binding FcγRIIIa at pH 6.5 versus the percent binding at pH 7.4 such that a value >1 indicates greater binding at pH 6.5. While the wild-type Fc exhibited a ratio of ˜1, indicating no pH selectivity, all variants showed increased pH-dependence, with 4A having the highest pH-dependence when measured on the CHO cell surface (FIG. 3C).

### Example 5—Characterization of Fc Variants as Soluble hu4D5 Antibodies

To assess pH selectivity in the context of purified protein, the four Fc variants were expressed as full-length human IgG1 antibodies with human anti-Her2 hu4D5 (also called Trastuzumab) Fab arms, observing similar yields as wild-type Fc. Binding of immobilized antibody to purified FcγRIIIa allele V158 was evaluated by ELISA at pH 6.5 and pH 7.4. No difference between binding at pH 6.5 and at pH 7.4 could be observed for wild-type Fc when compared on the same plate (FIG. 9C) but all variants showed greatly reduced FcγRIIIA binding versus wild-type at both pH values (FIG. 9D). This affinity loss was not apparent in the CHO display format, for several possible reasons. First, despite using a suboptimal Kozak sequence, avidity effects due to high Fc display could still mask reduced FcγRIIIa binding. Second, the Fc display construct included only the Fc domain to minimize plasmid size and increase transfection efficiency. The selected residue changes are near the hinge region and the presence of Fab arms may modulate FcγRIIIa access to this region.

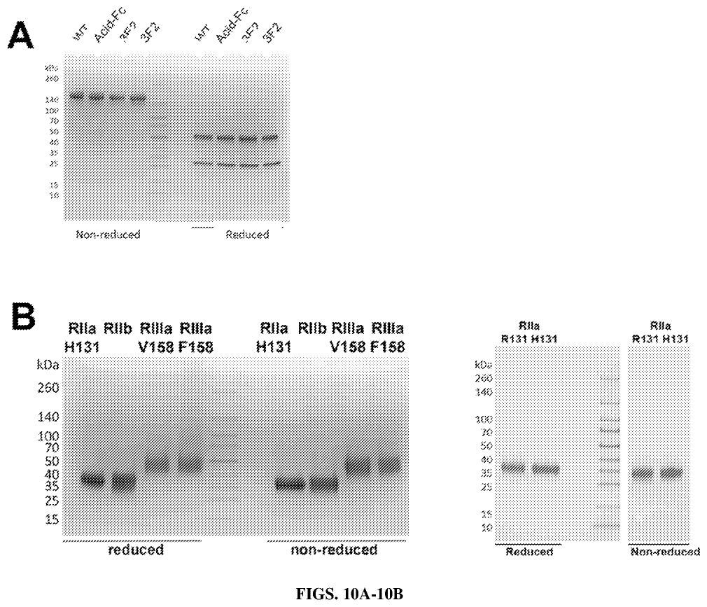

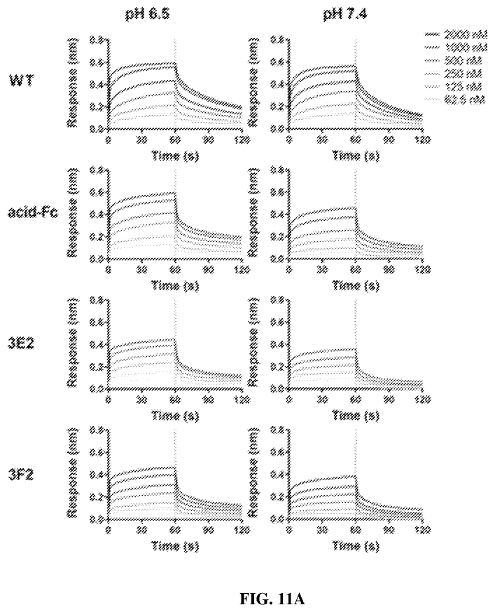

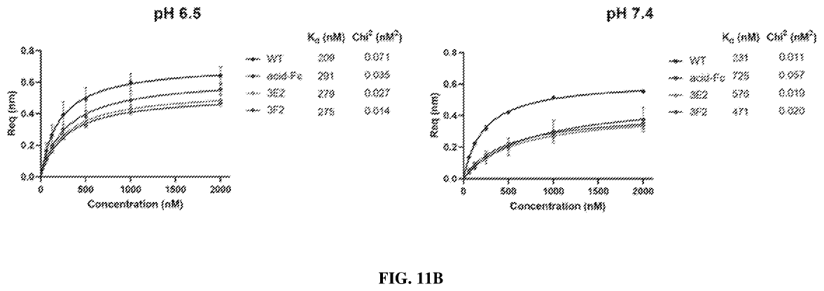

Further inspection of the selected sequences led to a consideration of whether E233, L234 and L235 in the lower hinge region could have different properties as a part of a full-length antibody versus a displayed Fc domain. The selected E233D and L234V substitutions are conservative changes shared among all four variants while L235 interacts with FcγRIIIa residues on both Fc chains (Sondermann et al., 2000; Mizushima et al., 2011). Therefore, it was speculated that reversion of these changes might recover binding affinity without losing pH dependence. Accordingly, a modified set of hu4D5 IgG variants (acid-Fc, 3E2, 3F2) with the native residues restored at positions 233-235 were generated by site-directed mutagenesis (Table 1; FIG. 10A). ELISA showed these new variants exhibit similar FcγRIIIa binding as wild-type at pH 6.5, as measured by the 50% effective concentration (EC₅₀), and reduced binding (larger EC₅₀) at pH 7.4, as predicted (FIG. 4A).

To provide a more quantitative assessment of the pH-selective FcγRIIIa binding of the selected Fc variants, biolayer interferometry (BLI), a technique that is particularly suitable for the moderate affinities of Fc-FcγR interactions, was used. The ectodomain of FcγRIIIa V158 was purified from Expi293 cells by immobilized metal chelate affinity chromatography (IMAC) (FIG. 10B). This protein was then enzymatically biotinylated and captured by streptavidin tips before dipping into wells containing one of the three hu4D5 Fc variants or wild-type Fc at each of six concentrations (62.5 nM to 2 μM) in pH 6.5 or pH 7.4 buffer (FIG. 11A) to determine steady-state apparent K_{d} values from Langmuir isotherms (FIG. 11B). All three Fc variants exhibited similar K_{d} values as the wild-type Fc for FcγRIIIa V158 at pH 6.5, and larger values than wild-type at pH 7.4. Among the three variants, acid-Fc had the highest K_{d,7.4}/K_{d,6.5} ratio of ˜2.6, indicating the greatest pH selectivity, and was selected for further investigation.

### Example 6—Acid-Fc Variant Exhibits pH-Selective FcγRIIa Binding

To characterize the pH-selective binding more carefully, the BLI experiment were repeated to collect kinetic data for hu4D5 with wild-type or acid-Fc binding to both FcγRIIIa alleles at both pH values. To allow for better regeneration of the biosensor tips, anti-CH1 FAB2G biosensors were used to capture each antibody and then the sensors were dipped into wells containing FcγRIIIa V158 or F158, at concentrations from 62.5 to 2000 nM and 156 to 5000 nM, respectively (FIG. 4B). Binding constants were calculated from on- and off-rates fitted to the entire association step and the initial dissociation step as suggested by the instrument manufacturer (ForteBio) for Fc/Fc receptor binding studies (Tobias et al., 2019). Antibodies bearing a wild-type Fc exhibited K_{d} values of 134±11.3 nM and 484±96 nM for FcγRIIIa V158 and F158 at pH 7.4, respectively, reflecting the expected affinity differences for these two allotypes previously measured by surface plasmon resonance (Ahmed et al., 2016; Bruhns et al., 2009). The measured K_{d} values for wild-type Fc at pH 6.5 appeared slightly, but not significantly, smaller for both alleles (˜15-30%). By contrast, K_{d} values for acid-Fc were ˜2-fold worse at pH 7.4 than pH 6.5 for both FcγRIIIa alleles (p<0.001) and also ˜2-fold worse than the values measured for wild-type Fc at pH 7.4 for each allele (p<0.001). The apparent K_{d,SS} values were also obtained by steady state analysis and agree well with kinetic values (FIG. 4C, Table 2). Due to the technical limitations of BLI measurements and the complex binding profiles of FcγR (Tobias et al., 2019; Kamat &Rafique, 2017), the reported K_{d} values are considered observed values for comparison between these Fc variants.

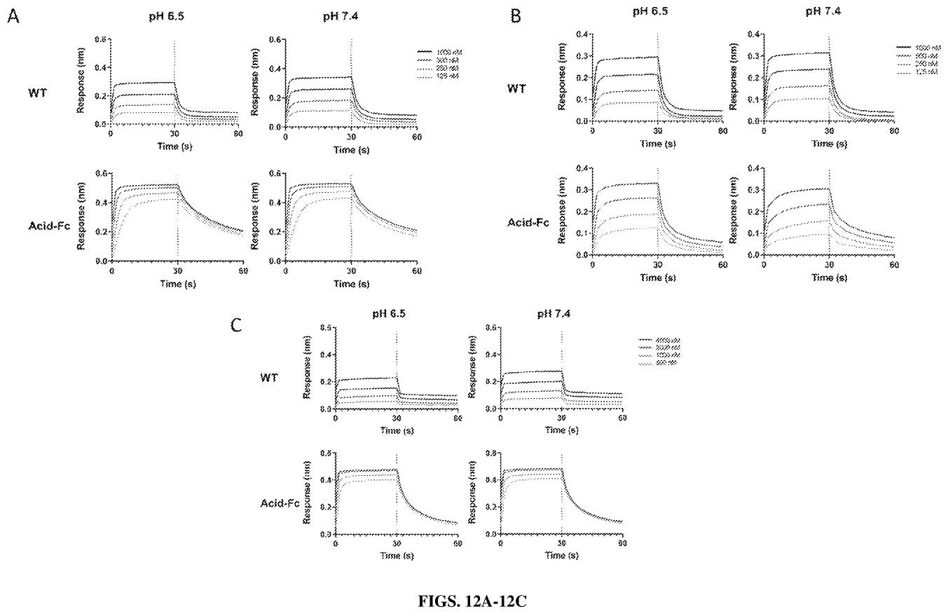

In addition to FcγRIIIa, other classical Fcγ receptors play important roles in anti-tumor efficacy (Ahmed et al., 2016; Nimmerjahn & Ravetch, 2006; Liu et al., 2020). To evaluate the effects of our engineered changes, BLI was used to measure binding of hu4D5 with wild-type Fc or acid-Fc to FcγRIIa H131, FcγRIIa R131 and FcγRIIb, using receptor ectodomains produced as above (FIG. 10B). FAB2G tips were again used to capture antibodies, which were then dipped into wells containing FcγRs at concentrations ranging from 125 nM to 4 μM in pH 6.5 or pH 7.4 buffer to determine equilibrium K_{d} values (FIG. 12, Table 5). Values for wild-type Fc binding at pH 7.4 were similar to previously reported values (Bruhns, 2012) with slightly weaker K_{d} values observed at pH 6.5. Acid-Fc exhibited significantly higher affinities for FcγRIIa (H131), FcγRIIa (R131) and FcγRIIb at both pH values. While wild-type showed some selectivity towards pH 7.4 for all three receptors, (K_{d,7.4}/K_{d,6.5} ratios of 0.67-0.79), acid-Fc showed no pH selectivity (K_{d,7.4}/K_{d,6.5} ratio ˜1) for FcγRIIa (R131) and FcγRIIb, and slight selectivity towards pH 6.5 for FcγRIIa (H131) with K_{d,7.4}/K_{d,6.5} ratio of ˜1.5.

TABLE 2
Binding affinity (K_{d}) to human FcγRIIIa, association (kₒₙ), dissociation (k_{off}) and equilibrium
binding (K_{d, SS}) constants of hu4D5 and selected Fc variants measured by BLI. Mean values and SD (n =
4) are shown, except for K_{d, SS} values for which the Chi2 values from the fit were shown.

| | | V158 | | | | | F158 | | | | |
|---|---|---|---|---|---|---|---|---|---|---|---|
| | | | | K_{d} ± | | | | | K_{d} ± | | |
| | | kₒₙ ± SD | k_{off} | SD | K_{d, SS} | Chi²_{Kd, SS} | kₒₙ ± SD | k_{off} | SD | K_{d, SS} | Chi²_{Kd, SS} |
| | | (×10⁵ M⁻¹s⁻¹) | (×10⁻²s⁻¹) | (nM) | (nM) | (nm²) | (×10⁵ M⁻¹s⁻¹) | (×10⁻²s⁻¹) | (nM) | (nM) | (nm²) |
| pH 6.5 | Wild- | 4.5 ± | 4.6 ± | 102 ± | 103 | 0.0303 | 3.0 ± | 11.9 ± | 401 ± | 418 | 0.0356 |
| | type | 1.1 | 1.2 | 2.4 | | | 3.2 | 1.1 | 74 | | |
| | Acid- | 5.7 ± | 7.4 ± | 131 ± | 115 | 0.0300 | 3.4 ± | 17.7 ± | 538 ± | 603 | 0.0298 |
| | Fc | 1.3 | 1.1 | 9.9 | | | 6.7 | 0.5 | 107 | | |
| pH 7.4 | Wild- | 4.9 ± | 6.4 ± | 134 ± | 139 | 0.0384 | 2.8 ± | 13.4 ± | 484 ± | 513 | 0.0445 |
| | type | 1.7 | 1.9 | 11.3 | | | 1.9 | 2.3 | 96 | | |
| | Acid- | 5.1 ± | 11 ± | 236 ± | 225 | 0.0297 | 2.5 ± | 22.6 ± | 909 ± | 1089 | 0.0347 |
| | Fc | 1.9 | 1.9 | 47 | | | 4.2 | 2.2 | 154 | | |

### Example 6—Acid-Fc Changes do not Impact Other Antibody Characteristics

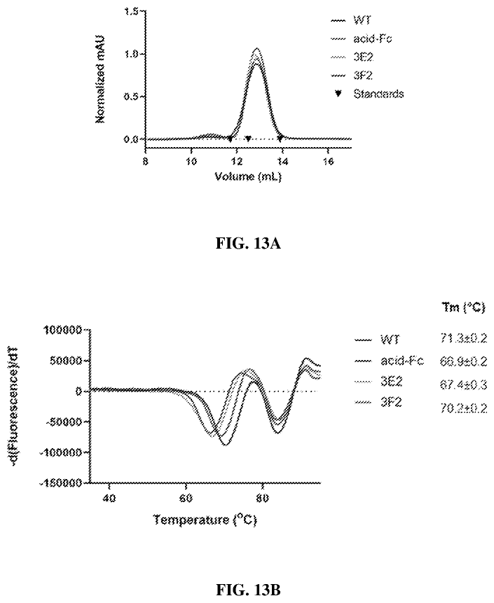

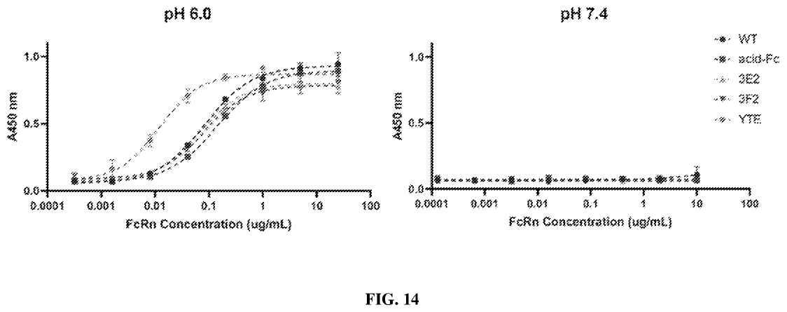

Fc engineering can introduce destabilizing and other undesirable effects, such as altered FcRn binding and pharmacokinetics (Liu et al., 2020). Accordingly, the biophysical characteristics of these new hu4D5 variants were evaluated. The observed molecular weights and sizes are similar to wild-type as assessed by SDS-PAGE gel and SEC (FIGS. 10A, 13A). All three variants were somewhat destabilized, as shown by lowered melting temperatures as compared to wild type (FIG. 13B). Variant 3F2 was the most thermo-stable with a decreased melting temperature of 1° C. versus wild-type, while acid-Fc exhibited a 4.4° C. loss.

Antibody in vivo half-life is largely determined by pH-selective binding between the Fc domain and FcRn. The acid-Fc mutations S267E, H268D, and Y296H are not in close contact (<5 Å) with FcRn or 02m residues in the co-crystal structure (Oganesyan et al., 2014) nor have changes at these locations been reported to impact FcRn binding. To provide an initial assessment of acid-Fc binding to FcRn, ELISA was used to assess binding of GST-tagged human FcRn-02m to an antibody-coated plate at pH 6.0 or pH 7.4. As expected, acid-Fc IgG showed similar binding to FcRn as wild-type 4D5 on ELISA (FIG. 13).

To provide a more rigorous assessment of FcRn binding behavior and evaluate the potential impacts of reduced acid-Fc thermostability in vivo, homozygous Tg32 mice, which express the human FcRn under the human promoter and are often used to evaluate antibody clearance rates, were used (Avery et al., 2016). Serum beta clearance of hu4D5 variants harboring wild-type, acid-Fc, and the M252Y/S254T/T256E (YTE) substitutions which extend in vivo half-life (Dall'Acqua et al., 2006) were assessed. Mice (n=4) were administered 2 mg/kg of each antibody intra-peritoneally and tail vein samples collected every ˜3 days. Serum antibody concentrations were determined by antigen-specific ELISA and plotted against time to determine the beta elimination half-life (FIG. 5). As expected, the YTE variant exhibited increased t_{1/2} as compared to wild-type (˜1.4-fold). Despite having a lowered melting temperature, acid-Fc showed a t_{1/2} of 9.5±2.3 days, similar to that observed for wild-type Fc (8.7±0.9 days). Power analysis indicates that groups of 118 would be required to detect differences between these two groups with confidence at α=0.05, suggesting the residue changes do not significantly impact in vivo stability.

To evaluate the impact of the increased affinity for FcγRIIa and FcγRIIb (FIG. 12, Table 5), the ability of hu4D5 with wild-type or acid-Fc to mediate antibody-dependent cellular phagocytosis (ADCP) was determined using a flow cytometric assay. This used the THP-1 monocytic human cell line that expresses FcγRI, FcγRIIa and FcγRIIb but not FcγRIIIa (Ackerman et al., 2011) and Her2-coated fluorescent beads that are also labeled with an intracellular pH fluorogenic probe. After incubation with cells and beads, both antibody variants mediated ADCP, with no significant different in their phagocytosis scores at the three antibody concentrations tested (FIG. 6A). Despite the changes in FcγRIIa/b affinities acid-Fc showed no significant change in ADCP activity, likely since the increased ratio of binding to activating/inhibitory receptors was off-set by differences in the numbers of receptors on each cell.

TABLE 5

| | K_{d, SS} ± SD (nM) | | | | | | | | |
|---|---|---|---|---|---|---|---|---|---|
| | FcγRIIa R131 | | | FcγRIIa H131 | | | FcγRIIb | | |
| 4D5 | | | Kd | pH | | Kd | | | Kd |
| variants | pH 6.5 | pH 7.4 | 7.4/Kd 6.5 | 6.5 | pH 7.4 | 7.4/Kd 6.5 | pH 6.5 | pH 7.4 | 7.4/Kd 6.5 |
| WT | 610 ± 20 | 411 ± 3 | 0.67 ± 0.03 | 549 ± 12 | 433 ± 3 | 0.79 ± 0.02 | 3411 ± 67 | 2562 ± 146 | 0.75 ± 0.06 |
| Acid-Fc | 37.1 ± 0.2 | 37.1 ± 0.4 | 1.00 ± 0.01 | 331 ± 6 | 497 ± 9 | 1.50 ± 0.03 | 112 ± 3 | 109 ± 3 | 0.97 ± 0.04 |

### Example 7—Acid-Fc Mediates pH Dependent ADCC Activity

To assess pH-selective activation of FcγRIIIa effector functions, a cell-based ADCC assay was performed. ADCC is triggered by binding of FcγRIIIa on an effector cell to clustered Fc domains whose Fab arms are bound to antigens on a target cell surface. Accordingly, ADCC was evaluated using Calcein-loaded SKOV3 ovarian carcinoma target cells and human NK-92 effector cells stably expressing FcγRIIIa (V158) (FIG. 6B). The antibody dose used (50 ng/mL) was determined to be within the dose response range for SKOV3 cells with moderate Her2 expression (˜10⁵/cell) (Lazar et al., 2006). In this experiment, hu4D5 with acid-Fc mediated lysis of ˜47.6±16.8% target cells at pH 6.5, similar to that achieved by wild-type Fc at pH 7.4 (49.8±5.9%) and pH 6.5 (39.1±3.2%). However, at pH 7.4, acid-Fc exhibited ˜2.4-fold reduced target cell lysis (19.5±5.6%, p<0.05), consistent with the pH-selective FcγRIIIa binding data.

To compare pH-selective ADCC activity more rigorously, the ADCC assays were repeated with SKBR3 target cells and NK-92/FcγRIIIa(V158) and effector cells in the presence of serially diluted antibody to assess the entire dose-response curve (FIG. 6C). SKBR3 is breast carcinoma cell line with high Her2 expression (˜10⁶/cell), characteristics of aggressive tumors (Lazar et al., 2006). Analysis of pooled data from multiple experiments shows minimal pH selectivity for wild-type Fc: EC₅₀ values are 96.28 ng/mL at pH 6.5 versus 101.4 ng/mL at pH 7.4, with overlapping 95% confidence intervals. Remarkably, the acid-Fc exhibited similar efficacy at pH 6.5 as the wild-type Fc, but ˜19-fold reduced activity at pH 7.4: EC₅₀ values of 120.7 ng/mL at pH 6.5 and 2307 ng/mL at pH 7.4, with non-overlapping 95% confidence intervals (Table 4).

TABLE 4
Four parameter logistic curves were fit to the ADCC dose-
response curves with GraphPad to obtain the EC₅₀ values
and 95% confidence intervals shown. Selectivity was calculated by
EC₅₀ at pH 7.4 over EC₅₀ at pH 6.5.

| | EC₅₀ (ng/mL) | | | | |
|---|---|---|---|---|---|
| 4D5 | | | | | Selectivity |
| variants | pH 6.5 | 95% CI | pH 7.4 | 95% CI | (pH 7.4/pH 6.5) |
| WT | 96.28 | 37.72 to | 101.4 | 30.24 to | 1.05 ± 1.03 |
| | | 200.8 | | 165.3 | |
| Acid-Fc | 120.7 | 25.99 to | 2307 | 813.7 to | 19.1 ± 2.5 |
| | | 437.0 | | 6926 | |

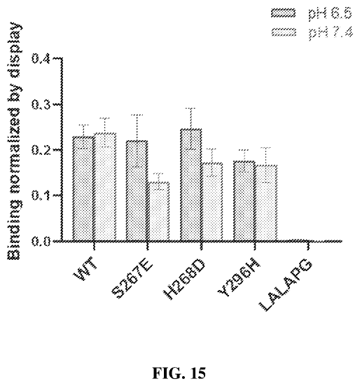

To better understand the relative contribution of selected residue changes to pH selectivity, the three acid-Fc changes (S267E, H268D and Y296H) were introduced individually into the wild-type Fc using site-directed mutagenesis. These single residue variants were then expressed on the CHO cell surface and assessed for FcγRIIIa binding at both pH values by flow cytometry (FIG. 15). The pH selectivity ratio defined as before is near one for wild-type and Y286, but is ˜20% higher for S267E and H268D (p<0.05; FIG. 7A), suggesting these two mutations contribute to pH-selectivity of acid-Fc through molecular interactions (FIG. 7B).

### Example 8—Acid-Fc Immune Checkpoint Inhibition

To assess the transferability of acid-Fc and its pH-selectivity to other cancer mAb therapeutics, anti-human CTLA-4 immune checkpoint inhibitor Ipilimumab (Yervoy), and anti-mouse CTLA-4 mAb (clone 4F10) were made with acid-Fc.

Jurkat cells were transfected with human mouse CTLA-4 and the ADCC activities were evaluated using NK-92 cells in vitro. Growth of F16B10 mouse melanoma cells in humanized FcγR mice will be evaluated in the presence of anti-CTLA-4 antibodies to evaluate efficacy with systemic cytokine responses used to evaluate on-tumor, off-target toxicities. We will also evaluate ex vivo ADCC activities and toxicities with human donor cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ackerman et al., A robust, high-throughput assay to determine the phagocytic activity of clinical antibody samples. J Immunol Methods 2011; 366:8-19.
Ahmed et al., Structural characterization of GASDALIE Fc bound to the activating Fc receptor FcγRIIIa. J. Struct. Biol. 194, 78-89 (2016).
Andreev et al., PH-sensitive membrane peptides (pHLIPs) as a novel class of delivery agents. Mol. Membr. Biol. 27, 341-352 (2010).
Avery et al., Utility of a human FcRn transgenic mouse model in drug discovery for early assessment and prediction of human pharmacokinetics of monoclonal antibodies. MAbs 8, 1064-1078 (2016).
Berntzen et al., Prolonged and increased expression of soluble Fc receptors, IgG and a TCR-Ig fusion protein by transiently transfected adherent 293E cells. J. Immunol. Methods 298, 93-104 (2005).
Bowles et al., Anti-CD20 monoclonal antibody with enhanced affinity for CD16 activates NK cells at lower concentrations and more effectively than rituximab. Blood 108, 2648-2654 (2006).
Brinkhaus et al., Glycine 236 in the Lower Hinge Region of Human IgG1 Differentiates Fc γ R from Complement Effector Function. (2021) doi:10.4049/jimmunol.2000961.
Bruhns et al., Specificity and affinity of human Fcγ receptors and their polymorphic variants for human IgG subclasses. Blood 113, 3716-3725 (2009).
Bruhns, Properties of mouse and human IgG receptors and their contribution to disease models. Blood 119, 5640-5649 (2012).
Challa et al., FcRn: From molecular interactions to regulation of IgG pharmacokinetics and functions. Curr. Top. Microbiol. Immunol. 382, 249-272 (2014).
Chen et al., Theranostics A general Fc engineering platform for the next generation of antibody therapeutics. 11, (2021).
Corbet & Feron, Tumour acidosis: From the passenger to the driver's seat. Nat. Rev. Cancer 17, 577-593 (2017).
Dall'Acqua et al., Properties of Human IgG1s engineered for enhanced binding to the neonatal Fc Receptor (FcRn). J. Biol. Chem. 281, 23514-23524 (2006).
Damaghi et al., pH sensing and regulation in cancer. Front. Physiol. 4 DEC, 1-10 (2013).
Delli Castelli et al., In Vivo Maps of Extracellular pH in Murine Melanoma by CEST-MRI. Magn Reson Med., 332, 326-332 (2014).
Eremina et al., VEGF Inhibition and Renal Thrombotic Microangiopathy. N. Engl. J. Med. 358, 1129-1136 (2008).
Fellouse et al., Synthetic antibodies from a four-amino-acid code: A dominant role for tyrosine in antigen recognition. Proc. Natl. Acad. Sci. U.S.A. 101, 12467-12472 (2004).
Ferreira et al., Tuning gene expression with synthetic upstream open reading frames. Proc. Natl. Acad. Sci. U.S.A. 110, 11284-9 (2013).
Force et al., Molecular mechanisms of cardiotoxicity of tyrosine kinase inhibition. Nat. Rev. Cancer 7, 332-344 (2007).
Forero-Torres et al., Results of a phase 1 study of AME-133v (LY2469298), an Fc-engineered humanized monoclonal anti-CD20 antibody, in FcγRIIIa-genotyped patients with previously treated follicular lymphoma. Clin. Cancer Res. 18, 1395-1403 (2012).
Goddard et al., UCSF ChimeraX: Meeting modern challenges in visualization and analysis. Protein Sci. 27, 14-25 (2018).
Gronwald et al., Cloning and expression of a cDNA coding for the human platelet-derived growth factor receptor: Evidence for more than one receptor class. Proc. Natl. Acad. Sci. U.S.A. 85, 3435-3439 (1988).
Hatjiharissi et al., Increased natural killer cell expression of CD16, augmented binding and ADCC activity to rituximab among individuals expressing the FcγRIIIa-158 V/V and V/F polymorphism. Blood 110, 2561-2564 (2007).
Holcmann & Sibilia, Mechanisms underlying skin disorders induced by EGFR inhibitors. Mol. Cell. Oncol. 2, (2015).
Jayapal et al., Recombinant protein therapeutics from CHO Cells—20 years and counting. Chem. Eng. Prog. 103, 40-47 (2007).
Johnston et al., VISTA is an acidic pH-selective ligand for PSGL-1. Nature 574, 565-570 (2019).
Jung et al., Effective phagocytosis of low Her2 tumor cell lines with engineered, aglycosylated igg displaying high FcγRIIa affinity and selectivity. ACS Chem. Biol. 8, 368-375 (2013).
Kamat & Rafique, Designing binding kinetic assay on the bio-layer interferometry (BLI) biosensor to characterize antibody-antigen interactions. Anal Biochem 2017; 536:16-31.
Kang et al., An Engineered Human Fc variant With Exquisite Selectivity for FcγRIIIaV158 Reveals That Ligation of FcγRIIIa Mediates Potent Antibody Dependent Cellular Phagocytosis With GM-CSF-Differentiated Macrophages. Front. Immunol. 10, 1-11 (2019).
Kato et al., Acidic extracellular microenvironment and cancer. Cancer Cell Int. 13, 1-8 (2013).
Kroetsch et al., Engineered pH-dependent recycling antibodies enhance elimination of Staphylococcal enterotoxin B superantigen in mice. MAbs 11, 411-421 (2019).
Kunaparaju et al., Epi-CHO, an episomal expression system for recombinant protein production in CHO cells. Biotechnol. Bioeng. 91, 670-677 (2005).
Lazar et al., Engineered antibody Fc variants with enhanced effector function. Proc. Natl. Acad. Sci. 103, 4005-4010 (2006).
Lee et al., An engineered human Fc domain that behaves like a pH-toggle switch for ultra-long circulation persistence. Nat. Commun. 10, 1-11 (2019).
Lee et al., IgG Fc domains that bind C1q but not effector Fcγ receptors delineate the importance of complement-mediated effector functions. Nat. Immunol. 18, 889-898 (2017).
Liu et al., Fc-Engineering for Modulated Effector Functions-Improving Antibodies for Cancer Treatment. Antibodies 9, 64 (2020).
Lo et al., Effector-attenuating substitutions that maintain antibody stability and reduce toxicity in mice. J. Biol. Chem. 292, 3900-3908 (2017).
Mellor et al., A critical review of the role of Fc gamma receptor polymorphisms in the response to monoclonal antibodies in cancer. J. Hematol. Oncol. 6, 1-10 (2013).
Mimoto et al., Novel asymmetrically engineered antibody Fc variant with superior FcγR binding affinity and specificity compared with afucosylated Fc variant. MAbs 5, 229-236 (2013).
Mizushima et al., Structural basis for improved efficacy of therapeutic antibodies on defucosylation of their Fc glycans. Genes to Cells 16, 1071-1080 (2011).
Moore et al., Engineered Fc variant antibodies with enhanced ability to recruit complement and mediate effector functions. MAbs 2, 181-189 (2010).
Mössner et al., Increasing the efficacy of CD20 antibody therapy through the engineering of a new type II anti-CD20 antibody with enhanced direct and immune effector cell-mediated B-cell cytotoxicity. Blood 115, 4393-4402 (2010).
Nguyen et al., Identification of high affinity HER2 binding antibodies using CHO Fab surface display. Protein Eng. Des. Sel. 31, 91-101 (2018).
Nimmerjahn & Ravetch, Fcγ receptors as regulators of immune responses. Nat. Rev. Immunol. 8, 34-47 (2008).
Nimmerjahn & Ravetch, Fcγ receptors: Old friends and new family members. Immunity 2006; 24:19-28.
Nimmerjahn & Ravetch, Translating basic mechanisms of IgG effector activity into next generation cancer therapies. Cancer Immun. 12, 13 (2012).
Nordstrom et al., Anti-tumor activity and toxicokinetics analysis of MGAH22, an anti-HER2 monoclonal antibody with enhanced Fcγ receptor binding properties. Breast Cancer Res. 13, 1-14 (2011).
Oganesyan et al., Structural insights into neonatal Fc receptor-based recycling mechanisms. J. Biol. Chem. 289, 7812-7824 (2014).
Overdijk et al., Crosstalk between Human IgG Isotypes and Murine Effector Cells. J. Immunol. 189, 3430-3438 (2012).
Pettersen et al., UCSF ChimeraX: Structure visualization for researchers, educators, and developers. Protein Sci. 30, 70-82 (2021).
Piccart-Gebhart et al., Trastuzumab after Adjuvant Chemotherapy in HER2-Positive Breast Cancer. N. Engl. J. Med. 353, 1659-1672 (2005).
Rugo et al., Efficacy of Margetuximab vs Trastuzumab in Patients with Pretreated ERBB2-Positive Advanced Breast Cancer: A Phase 3 Randomized Clinical Trial. JAMA Oncol. 7, 573-584 (2021).
Sarkar et al., Rational cytokine design for increased lifetime and enhanced potency using pH-activated “histidine switching”. Nat. Biotechnol. 20, 908-913 (2002).
Sazinsky et al., Aglycosylated immunoglobulin G1 variants productively engage activating Fc receptors. Proc. Natl. Acad. Sci. U.S.A. 105, 20167-20172 (2008).
Scott et al., Monoclonal antibodies in cancer therapy. Cancer Immun. 12, 14 (2012).
Shields et al., High Resolution Mapping of the Binding Site on Human IgG1 for FcγRI, FcγRII, FcγRIII, and FcRn and Design of IgG1 Variants with Improved Binding to the FcγR. J. Biol. Chem. 276, 6591-6604 (2001).
Sondermann et al., The 3.2-A crystal structure of the human IgG1 Fc fragment-FcγRIII complex. Nature 406, 267-273 (2000).
Stavenhagen et al., Fc optimization of therapeutic antibodies enhances their ability to kill tumor cells in vitro and controls tumor expansion in vivo via low-affinity activating Fcγ receptors. Cancer Res 2007; 67:8882-90.
Sulea et al., Structure-based engineering of pH-dependent antibody binding for selective targeting of solid-tumor microenvironment. MAbs 12, (2020).
Tobias et al., Analysis of FcRn-Antibody Interactions on the Octet Platform. ForteBio Appl Note 17, pp. 1-13, 2019; Available on the world wide web at: fortebio.com/sites/default/files/en/assets/app-note/analysis-of-fc-gamma-receptor-igg-interactions-on-octet-platform.pdf
Wagner et al., Human cytomegalovirus-specific T-cell receptor engineered for high affinity and soluble expression using mammalian cell display. J. Biol. Chem. 294, 5790-5804 (2019).
Warburg, On the origin of cancer cells. Science (80-.). 123, 309-314 (1956).
Webb et al., Inhibition of MMP-2 and MMP-9 decreases cellular migration, and angiogenesis in in vitro models of retinoblastoma. BMC Cancer 17, 1-11 (2017).
Webb et al., Dysregulated pH: A perfect storm for cancer progression. Nat. Rev. Cancer 11, 671-677 (2011).
Wei et al., Mapping pH at Cancer Cell Surfaces. Mol. Imaging Biol. 21, 1020-1025 (2019).
Wike-Hooley et al., The relevance of tumour pH to the treatment of malignant disease. Radiother. Oncol. 2, 343-66 (1984).

1. A recombinant polypeptide comprising
a pH-selective mammalian IgG1 Fc domain, wherein the pH-selective IgG1 Fc domain has higher affinity for FcγRIIIa at pH 6.5 than at pH 7.4, wherein the pH-selective mammalian IgG1 Fc domain comprises a substitution at one or more of the following positions: H268, S267, Y296, and/or S298, wherein the positions are numbered according to the EU numbering system.

2-3. (canceled)

4. The recombinant polypeptide of claim 1, wherein the pH-selective mammalian IgG1 Fc domain has an affinity for FcγRIIIa at neutral pH that is at least about 3-fold lower than the affinity of a wild-type mammalian IgG1 Fc domain for FcγRIII.

5. The recombinant polypeptide of claim 1, wherein the pH-selective mammalian IgG1 Fc domain has an equivalent affinity for FcγRIIIa at pH 6.5 as compared to a wild-type mammalian IgG1 Fc domain.

6. The recombinant polypeptide of claim 1, wherein the pH-selectivity is determined as the ratio of the affinity for FcγRIIIa at pH 6.5 versus the affinity at pH 7.4.

7. The recombinant polypeptide of claim 1, wherein the pH-selective mammalian IgG1 Fc domain has an equivalent affinity for FcRn as a wild-type mammalian IgG1 Fc domain.

8-9. (canceled)

10. The recombinant polypeptide of claim 1, wherein the pH-selective mammalian IgG1 Fc domain has ADCC activity at neutral pH that is at least about 3-fold lower than the ADCC activity of a wild-type mammalian IgG1 Fc domain.

11-12. (canceled)

13. The recombinant polypeptide of claim 1, wherein the pH-selective mammalian IgG1 Fc domain comprises an amino acid sequence of SEQ ID NO: 3.

14. The recombinant polypeptide of claim 1, wherein the pH-selective mammalian IgG1 Fc domain comprises the following substitutions: S267E, H268D, and Y296H, wherein the positions are numbered according to the EU numbering system.

15. The recombinant polypeptide of claim 14, wherein the pH-selective mammalian IgG1 Fc domain comprises an amino acid sequence of SEQ ID NO: 4.

16. The recombinant polypeptide of claim 1, wherein the pH-selective mammalian IgG1 Fc domain comprises the following substitutions: S267G, H268D, and Y296H, wherein the positions are numbered according to the EU numbering system.

17. The recombinant polypeptide of claim 15, wherein the pH-selective mammalian IgG1 Fc domain comprises an amino acid sequence of SEQ ID NO: 5.

18. The recombinant polypeptide of claim 1, wherein the pH-selective mammalian IgG1 Fc domain comprises the following substitutions: S267D, H268D, and S298R, wherein the positions are numbered according to the EU numbering system.

19. The recombinant polypeptide of claim 18, wherein the pH-selective mammalian IgG1 Fc domain comprises an amino acid sequence of SEQ ID NO: 6.

20. The recombinant polypeptide of claim 1, wherein the pH-selective mammalian IgG1 Fc domain comprises an amino acid sequence at least 90% identical to SEQ ID NO: 1 or 2.

21. (canceled)

22. The recombinant polypeptide of claim 1, wherein the pH-selective mammalian IgG1 Fc domain is glycosylated at residue N297, wherein the position is numbered according to the EU numbering system.

23-40. (canceled)

41. An isolated nucleic acid encoding the recombinant polypeptide of claim 1.

42. An expression vector comprising the nucleic acid of claim 41.

43. A hybridoma or engineered cell comprising a nucleic acid encoding the recombinant polypeptide of claim 1.

44-45. (canceled)

46. A pharmaceutical formulation comprising one or more recombinant polypeptide of claim 1.

47. A method of treating a subject comprising administering an effective amount of the pharmaceutical formulation of claim 46 to the subject.

48-52. (canceled)
